SEQUENCE LISTING (80 sequences)

SEQ ID NO: 1 - DNA, 21 bp, Artificial Sequence
  Description of Artificial Sequence Synthetic primer
cttcatcctgctgctatgcct

SEQ ID NO: 2 - DNA, 20 bp, Artificial Sequence
  Description of Artificial Sequence Synthetic primer
cccaactcctcccagtcctt

SEQ ID NO: 3 - DNA, 23 bp, Artificial Sequence
  Description of Artificial Sequence Synthetic primer
gtttaattttcggtttcgtcgtc

SEQ ID NO: 4 - DNA, 17 bp, Artificial Sequence
  Description of Artificial Sequence Synthetic primer
ctcccgacgtcgctacg

SEQ ID NO: 5 - DNA, 21 bp, Artificial Sequence
  Description of Artificial Sequence Synthetic primer
cggttttcgttcgttttttcg

SEQ ID NO: 6 - DNA, 20 bp, Artificial Sequence
  Description of Artificial Sequence Synthetic primer
gtaacttccgccttctacgc

SEQ ID NO: 7 - DNA, 16 bp, Artificial Sequence
  Description of Artificial Sequence Synthetic primer
ggcggttcgggtatcg

SEQ ID NO: 8 - DNA, 21 bp, Artificial Sequence
  Description of Artificial Sequence Synthetic primer
cgtaatcacgtaactccgact

SEQ ID NO: 9 - DNA, 18 bp, Artificial Sequence
  Description of Artificial Sequence Synthetic primer
tcgttgggtaaggcgttc

SEQ ID NO: 10 - DNA, 18 bp, Artificial Sequence
  Description of Artificial Sequence Synthetic primer
aaacgaacacccgaaccg

SEQ ID NO: 11 - DNA, 27 bp, Artificial Sequence
  Description of Artificial Sequence Synthetic primer
tttgtttttttgattaggtgtttaaga

SEQ ID NO: 12 - DNA, 21 bp, Artificial Sequence
  Description of Artificial Sequence Synthetic primer
caccaacctcataaccttatc

SEQ ID NO: 13 - DNA, 70 bp, Artificial Sequence
  Description of Artificial Sequence Synthetic oligonucleotide
  modified_base (22)..(29): a, c, t, g, unknown or other
cttcatcctgctgctatgcctnnnnnnnncaccaacctcataaccttatcacacaaaccaatattaatac

SEQ ID NO: 14 - DNA, 64 bp, Artificial Sequence
  Description of Artificial Sequence Synthetic oligonucleotide
ctacacccacaacactatcttaaacacctaatcaaaaaaacaaaaaggactgggaggagttggg

SEQ ID NO: 15 - DNA, 63 bp, Artificial Sequence
  Description of Artificial Sequence Synthetic oligonucleotide
  modified_base (22)..(29): a, c, t, g, unknown or other
cttcatcctgctgctatgcctnnnnnnnnacgtaatcacgtaactccgactaaaactcgaccg

SEQ ID NO: 16 - DNA, 46 bp, Artificial Sequence
  Description of Artificial Sequence Synthetic oligonucleotide
actcgcgatacccgaaccgccgaacaaaggactgggaggagttggg

SEQ ID NO: 17 - DNA, 62 bp, Artificial Sequence
  Description of Artificial Sequence Synthetic oligonucleotide
  modified_base (22)..(29): a, c, t, g, unknown or other
cttcatcctgctgctatgcctnnnnnnnnataacttccgccttctacgcgactaaaataccc

SEQ ID NO: 18 - DNA, 51 bp, Artificial Sequence
  Description of Artificial Sequence Synthetic oligonucleotide
gataaacgaacgaaaaaacgaacgaaaaccgaaggactgggaggagttggg

SEQ ID NO: 19 - DNA, 62 bp, Artificial Sequence
  Description of Artificial Sequence Synthetic oligonucleotide
  modified_base (22)..(29): a, c, t, g, unknown or other
cttcatcctgctgctatgcctnnnnnnnnctcccgacgtcgctacgaaacactccgaaaacg

SEQ ID NO: 20 - DNA, 52 bp, Artificial Sequence
  Description of Artificial Sequence Synthetic oligonucleotide
caaaaaaccgacgacgaaaccgaaaattaaacaaggactgggaggagttggg

SEQ ID NO: 21 - DNA, 66 bp, Artificial Sequence
  Description of Artificial Sequence Synthetic oligonucleotide
  modified_base (22)..(29): a, c, t, g, unknown or other
cttcatcctgctgctatgcctnnnnnnnnaaacgaacacccgaaccgcctaaaacaaaaaaccgcg

SEQ ID NO: 22 - DNA, 44 bp, Artificial Sequence
  Description of Artificial Sequence Synthetic oligonucleotide
cacctcctcccgccaaacgctttctcgaacgccttacccaacga

SEQ ID NO: 23 - DNA, 50 bp, Artificial Sequence
  Description of Artificial Sequence Synthetic oligonucleotide
  modified_base (22)..(29): a, c, t, g, unknown or other
cttcatcctgctgctatgcctnnnnnnnncaccgccgccgcgcgctctac

SEQ ID NO: 24 - DNA, 50 bp, Artificial Sequence
  Description of Artificial Sequence Synthetic oligonucleotide
gcctacaaaaattaaacgacaacgcacgcgaaggactgggaggagttggg

SEQ ID NO: 25 - DNA, 59 bp, Artificial Sequence
  Description of Artificial Sequence Synthetic oligonucleotide
  modified_base (22)..(29): a, c, t, g, unknown or other
cttcatcctgctgctatgcctnnnnnnnnccgaacgccccgctacgaccaaatataaac

SEQ ID NO: 26 - DNA, 59 bp, Artificial Sequence
  Description of Artificial Sequence Synthetic oligonucleotide
gaatataaaaaccgaaccataacgaaaaaaacgaacgccaaggactgggaggagttggg

SEQ ID NO: 27 - DNA, 48 bp, Artificial Sequence
  Description of Artificial Sequence Synthetic oligonucleotide
  modified_base (22)..(29): a, c, t, g, unknown or other
cttcatcctgctgctatgcctnnnnnnnntcctccccgcctaacccgc

SEQ ID NO: 28 - DNA, 47 bp, Artificial Sequence
  Description of Artificial Sequence Synthetic oligonucleotide
gcccaaaaaacgacgaaaaacgcgaccaaggactgggaggagttggg

SEQ ID NO: 29 - DNA, 55 bp, Artificial Sequence
  Description of Artificial Sequence Synthetic oligonucleotide
  modified_base (22)..(29): a, c, t, g, unknown or other
cttcatcctgctgctatgcctnnnnnnnncccgaacgaatcaaattcccgcaccc

SEQ ID NO: 30 - DNA, 44 bp, Artificial Sequence
  Description of Artificial Sequence Synthetic oligonucleotide
gcaccgacctccctatctcgcactaaggactgggaggagttggg

SEQ ID NO: 31 - DNA, 54 bp, Artificial Sequence
  Description of Artificial Sequence Synthetic oligonucleotide
  modified_base (22)..(29): a, c, t, g, unknown or other
cttcatcctgctgctatgcctnnnnnnnnaaaacgaacgccgacccctccccgc

SEQ ID NO: 32 - DNA, 39 bp, Artificial Sequence
  Description of Artificial Sequence Synthetic oligonucleotide
gcgctacgcccccgccccgaaggactgggaggagttggg

SEQ ID NO: 33 - DNA, 53 bp, Artificial Sequence
  Description of Artificial Sequence Synthetic oligonucleotide
  modified_base (22)..(29): a, c, t, g, unknown or other
cttcatcctgctgctatgcctnnnnnnnncgcgctaactaaaacgccgcgccc

SEQ ID NO: 34 - DNA, 50 bp, Artificial Sequence
  Description of Artificial Sequence Synthetic oligonucleotide
gcgctcctacaatacaaaactaaccgccgaaaggactgggaggagttggg

SEQ ID NO: 35 - DNA, 57 bp, Artificial Sequence
  Description of Artificial Sequence Synthetic oligonucleotide
  modified_base (22)..(29): a, c, t, g, unknown or other
cttcatcctgctgctatgcctnnnnnnnnacaacgaatcgcgcgaaaaacaacgacg

SEQ ID NO: 36 - DNA, 44 bp, Artificial Sequence
  Description of Artificial Sequence Synthetic oligonucleotide
aaaaaacgcccccgacgaaacccgaaggactgggaggagttggg

SEQ ID NO: 37 - DNA, 50 bp, Artificial Sequence
  Description of Artificial Sequence Synthetic oligonucleotide
  modified_base (22)..(29): a, c, t, g, unknown or other
cttcatcctgctgctatgcctnnnnnnnncgtcccgcgccaaacccaccc

SEQ ID NO: 38 - DNA, 50 bp, Artificial Sequence
  Description of Artificial Sequence Synthetic oligonucleotide
gcaaaatcctctccaacacgtccgcgaccgaaggactgggaggagttggg

SEQ ID NO: 39 - DNA, 60 bp, Artificial Sequence
  Description of Artificial Sequence Synthetic oligonucleotide
  modified_base (22)..(29): a, c, t, g, unknown or other
cttcatcctgctgctatgcctnnnnnnnncgtaaacgacgcgaacacgaaaccgaaaacg

SEQ ID NO: 40 - DNA, 46 bp, Artificial Sequence
  Description of Artificial Sequence Synthetic oligonucleotide
cgcgacgctctcaacgaaaaaacgccaaggactgggaggagttggg

SEQ ID NO: 41 - DNA, 63 bp, Artificial Sequence
  Description of Artificial Sequence Synthetic oligonucleotide
  modified_base (22)..(29): a, c, t, g, unknown or other
cttcatcctgctgctatgcctnnnnnnnncgaaaataaaaaacgacctccctatcccgttacc

SEQ ID NO: 42 - DNA, 48 bp, Artificial Sequence
  Description of Artificial Sequence Synthetic oligonucleotide
gaatccaaacgaaacccgaaaaccgccgaaggactgggaggagttggg

SEQ ID NO: 43 - DNA, 65 bp, Artificial Sequence
  Description of Artificial Sequence Synthetic oligonucleotide
  modified_base (22)..(29): a, c, t, g, unknown or other
cttcatcctgctgctatgcctnnnnnnnnaaacgaacacccgaaccgcctaaaacaaaaaaccgc

SEQ ID NO: 44 - DNA, 52 bp, Artificial Sequence
  Description of Artificial Sequence Synthetic oligonucleotide
gcacctcctcccgccaaacgctttctcgaacgaaggactgggaggagttggg

SEQ ID NO: 45 - DNA, 64 bp, Artificial Sequence
  Description of Artificial Sequence Synthetic oligonucleotide
  modified_base (22)..(29): a, c, t, g, unknown or other
cttcatcctgctgctatgcctnnnnnnnnaccacgacctaaactatatccgttcgatactcccg

SEQ ID NO: 46 - DNA, 51 bp, Artificial Sequence
  Description of Artificial Sequence Synthetic oligonucleotide
acccacgaactacacaacttaacgaccccgaaaggactgggaggagttggg

SEQ ID NO: 47 - DNA, 59 bp, Artificial Sequence
  Description of Artificial Sequence Synthetic oligonucleotide
  modified_base (22)..(29): a, c, t, g, unknown or other
cttcatcctgctgctatgcctnnnnnnnncgcgaacgcaaaacgcaaaaccccaacacc

SEQ ID NO: 48 - DNA, 56 bp, Artificial Sequence
  Description of Artificial Sequence Synthetic oligonucleotide
gacaatcaaaaaaacgcaaaaaaacgcacgcactcaaaggactgggaggagttggg

SEQ ID NO: 49 - DNA, 59 bp, Artificial Sequence
  Description of Artificial Sequence Synthetic oligonucleotide
  modified_base (22)..(29): a, c, t, g, unknown or other
cttcatcctgctgctatgcctnnnnnnnnccaaacgacaacgacctcctcgaaaactcg

SEQ ID NO: 50 - DNA, 52 bp, Artificial Sequence
  Description of Artificial Sequence Synthetic oligonucleotide
cgaaactacctcgaaactctccaaaacgcacaaaggactgggaggagttggg

SEQ ID NO: 51 - DNA, 52 bp, Artificial Sequence
  Description of Artificial Sequence Synthetic oligonucleotide
  modified_base (22)..(29): a, c, t, g, unknown or other
cttcatcctgctgctatgcctnnnnnnnncgctaaaaacgccgcgcgccccc

SEQ ID NO: 52 - DNA, 40 bp, Artificial Sequence
  Description of Artificial Sequence Synthetic oligonucleotide
gaccccgtacccgcgccgccaaggactgggaggagttggg

SEQ ID NO: 53 - DNA, 51 bp, Artificial Sequence
  Description of Artificial Sequence Synthetic oligonucleotide
  modified_base (22)..(29): a, c, t, g, unknown or other
cttcatcctgctgctatgcctnnnnnnnnccgccgaaaacgcttcctcgcc

SEQ ID NO: 54 - DNA, 43 bp, Artificial Sequence
  Description of Artificial Sequence Synthetic oligonucleotide
gctaccctccgcgcgacccgctaaaggactgggaggagttggg

SEQ ID NO: 55 - DNA, 64 bp, Artificial Sequence
  Description of Artificial Sequence Synthetic oligonucleotide
  modified_base (22)..(33): a, c, t, g, unknown or other
cttcatcctgctgctatgcctnnnnnnnnnnnnactcctctacatacgccgcgaataaattacc

SEQ ID NO: 56 - DNA, 48 bp, Artificial Sequence
  Description of Artificial Sequence Synthetic oligonucleotide
gaaaacatcgaccgaacaacgacgtccgaaggactgggaggagttggg

SEQ ID NO: 57 - DNA, 60 bp, Artificial Sequence
  Description of Artificial Sequence Synthetic oligonucleotide
  modified_base (22)..(33): a, c, t, g, unknown or other
cttcatcctgctgctatgcctnnnnnnnnnnnncgtcctactaacgaccgacgctccaac

SEQ ID NO: 58 - DNA, 39 bp, Artificial Sequence
  Description of Artificial Sequence Synthetic oligonucleotide
gtacgaaaacgcgccgcctaaggactgggaggagttggg

SEQ ID NO: 59 - DNA, 56 bp, Artificial Sequence
  Description of Artificial Sequence Synthetic oligonucleotide
  modified_base (22)..(33): a, c, t, g, unknown or other
cttcatcctgctgctatgcctnnnnnnnnnnnngcccctcttccgcgcgtaacccc

SEQ ID NO: 60 - DNA, 42 bp, Artificial Sequence
  Description of Artificial Sequence Synthetic oligonucleotide
gaaccgcgaatacgaccgcgacaaggactgggaggagttggg

SEQ ID NO: 61 - DNA, 63 bp, Artificial Sequence
  Description of Artificial Sequence Synthetic oligonucleotide
  modified_base (22)..(33): a, c, t, g, unknown or other
cttcatcctgctgctatgcctnnnnnnnnnnnncgaccgcgaaaaccacaaataaactcgctc

SEQ ID NO: 62 - DNA, 50 bp, Artificial Sequence
  Description of Artificial Sequence Synthetic oligonucleotide
gcaaacgctaacccgaataaaactaaacgaaaggactgggaggagttggg

SEQ ID NO: 63 - DNA, 56 bp, Artificial Sequence
  Description of Artificial Sequence Synthetic oligonucleotide
  modified_base (22)..(33): a, c, t, g, unknown or other
cttcatcctgctgctatgcctnnnnnnnnnnnnaaacgccccgcaacccgacaacc

SEQ ID NO: 64 - DNA, 43 bp, Artificial Sequence
  Description of Artificial Sequence Synthetic oligonucleotide
gcctctcgaactctaccgcccgcaaggactgggaggagttggg

SEQ ID NO: 65 - DNA, 67 bp, Artificial Sequence
  Description of Artificial Sequence Synthetic oligonucleotide
  modified_base (22)..(33): a, c, t, g, unknown or other
cttcatcctgctgctatgcctnnnnnnnnnnnncgcgaaaaccgaacaacgaactactaaacatccc

SEQ ID NO: 66 - DNA, 49 bp, Artificial Sequence
  Description of Artificial Sequence Synthetic oligonucleotide
gcgaacgacgacaacaaaaacgacgacgaaaggactgggaggagttggg

SEQ ID NO: 67 - DNA, 68 bp, Artificial Sequence
  Description of Artificial Sequence Synthetic oligonucleotide
  modified_base (22)..(33): a, c, t, g, unknown or other
cttcatcctgctgctatgcctnnnnnnnnnnnncgacttaacccgacgatcgcgacgaaattcctaac

SEQ ID NO: 68 - DNA, 56 bp, Artificial Sequence
  Description of Artificial Sequence Synthetic oligonucleotide
gcaaccgcttaaaacttcgcattaaaatcgaaaccgaaggactgggaggagttggg

SEQ ID NO: 69 - DNA, 71 bp, Artificial Sequence
  Description of Artificial Sequence Synthetic oligonucleotide
  modified_base (22)..(33): a, c, t, g, unknown or other
cttcatcctgctgctatgcctnnnnnnnnnnnncgctacgaacgcacataaccaaataacgcctaacaaac

SEQ ID NO: 70 - DNA, 51 bp, Artificial Sequence
  Description of Artificial Sequence Synthetic oligonucleotide
gacgtccgaatacaaaacgacgctcttaccgaaggactgggaggagttggg

SEQ ID NO: 71 - DNA, 63 bp, Artificial Sequence
  Description of Artificial Sequence Synthetic oligonucleotide
  modified_base (22)..(33): a, c, t, g, unknown or other
cttcatcctgctgctatgcctnnnnnnnnnnnnacccgcgtctaaacaacgtctctaaccaac

SEQ ID NO: 72 - DNA, 52 bp, Artificial Sequence
  Description of Artificial Sequence Synthetic oligonucleotide
gacgttaatcaacaaacgtacccgcgatcgctaaggactgggaggagttggg

SEQ ID NO: 73 - DNA, 62 bp, Artificial Sequence
  Description of Artificial Sequence Synthetic oligonucleotide
  modified_base (22)..(33): a, c, t, g, unknown or other
cttcatcctgctgctatgcctnnnnnnnnnnnncgcacaaccatcccaaaccgaacaaccgc

SEQ ID NO: 74 - DNA, 46 bp, Artificial Sequence
  Description of Artificial Sequence Synthetic oligonucleotide
gcgccgaaacgaaaacgaaaaccgcaaaggactgggaggagttggg

SEQ ID NO: 75 - DNA, 52 bp, Artificial Sequence
  Description of Artificial Sequence Synthetic oligonucleotide
  modified_base (22)..(33): a, c, t, g, unknown or other
cttcatcctgctgctatgcctnnnnnnnnnnnnccgacgcgacccgccaacc

SEQ ID NO: 76 - DNA, 38 bp, Artificial Sequence
  Description of Artificial Sequence Synthetic oligonucleotide
gacccgccgaaacgcgctaaggactgggaggagttggg

SEQ ID NO: 77 - DNA, 60 bp, Artificial Sequence
  Description of Artificial Sequence Synthetic oligonucleotide
tgttcggcggttcgggtatcgcgagtcggtcgagttttagtcggagttacgtgattacgt

SEQ ID NO: 78 - DNA, 26 bp, Artificial Sequence
  Description of Artificial Sequence Synthetic oligonucleotide
actcgcgatacccgaaccgccgaaca

SEQ ID NO: 79 - DNA, 34 bp, Artificial Sequence
  Description of Artificial Sequence Synthetic oligonucleotide
acgtaatcacgtaactccgactaaaactcgaccg

SEQ ID NO: 80 - DNA, 60 bp, Artificial Sequence
  Description of Artificial Sequence Synthetic oligonucleotide
tgtttggtggtttgggtattgtgagttggttgagttttagttggagttatgtgattatgt

(19) United States
(12) Patent Application Publication
(10) Pub. No.: US 2021/0095341 A1
(43) Pub. Date: Apr. 1, 2021

(54) MULTIPLEX 5MC MARKER BARCODE COUNTING FOR METHYLATION DETECTION IN CELL FREE DNA

(71) Applicant: The University of Chicago, Chicago, IL (US)

(72) Inventors: Chuan HE, Chicago, IL (US); Ji NIE, Chicago, IL (US)

(21) Appl. No.: 15/733,262

(22) PCT Filed: Dec. 19, 2018
(86) PCT No.: PCT/US2018/066485

§ 371 Date: Jun. 18, 2020

Related U.S. Application Data
(60) Provisional application No. 62/609,922, filed on Dec. 22, 2017.

(51) Int. Cl. C12Q 1/6883; C12Q 1/6827; C12Q 1/686

(52) U.S. Cl. CPC C12Q 1/6883; C12Q 1/6827; C12Q 1/686; C12Q 2600/166; C12Q 2600/154; C12Q 2600/16; C12Q 2525/161; C12Q 2531/107; C12Q 2523/125; C12Q 2521/319; C12Q 2521/501; C12Q 2521/539; C12Q 2563/179; C12Q 2563/125

(57) ABSTRACT

Described herein is a multiplex 5mC marker barcode counting (MMBC) to quantify 5mC markers in DNA, in which multiple 5mC markers could be targeted for capture and linear amplification. The inventors' method provides highly sensitive and specific quantification of multiple 5mC loci. Accordingly, aspects of the disclosure relate to a method for detecting methylated or unmethylated cytosines in one or more regions of target nucleic acids, the method comprising i) combining a solution comprising the target nucleic acids with a deaminating agent to convert unmethylated cytosines in the target nucleic acids to uracils; ii) next contacting the solution with at least two probes under conditions that allow for the hybridization of the two probes to one target nucleic acid region; wherein a terminal end from each probe hybridizes adjacently to the target nucleic acid region; iii) contacting the solution comprising the hybridized probes and target nucleic acids with a ligase under conditions that allow for the ligation of the terminal ends of the adjacently hybridized probes; and iv) detecting the adjacently hybridized ligated probes in the solution.

### CROSS-REFERENCE TO RELATED APPLICATIONS

This application claims the benefit of priority of U.S. Provisional Patent Application No. 62/609,922 filed Dec. 22, 2017, which is hereby incorporated by reference in its entirety.

### BACKGROUND

### 1. Field of the Invention

The current disclosure relates to cell biology techniques that can be used in diagnostic and research applications.

### 2. Description of Related Art

Cell-free DNAs (cfDNA) are short DNA fragments in circulating plasma, urine, and other body fluids that are released from apoptotic cells. [1] Because malignant tumor cells are known to undergo apoptosis and can release a variable proportion of cfDNA into the circulating blood, sensitive detections of biomarkers associated with tumor-specific cfDNA fragments have the potentials to offer non-invasion, convenient, and cost-effective approaches to diagnose and monitor the initiation and progression of human cancer with much better patient compliance. [2] DNA methylation (5-methylcytosine, 5mC) is an epigenetic mechanism that plays critical roles in a wide range of biological processes.[3] Aberrant DNA methylation could reflect gene expression changes and the occurrence and development of human disease. Apart from cancer-specific mutations, DNA methylation status provides epigenetic information, which offers an alternative approach in cancer diagnosis and prognosis using cfDNA. In the past, locus-specific assays, such as methylation-specific PCR (MSP), bisulfite sequencing PCR (BSP), MethyLight etc.,[4] have been developed and implemented for detection of hypermethylation loci in clinical samples (e.g., SEPT9 methylation for colon cancer screening) due to their low cost, simplicity, and relatively high sensitivity.[5] However, these PCR-based assays only target one or limited 5mC markers in a single reaction; more comprehensive information carried by multiple 5mC-bearing loci in cfDNA from malignant cells could not be obtained, therefore hampering the accuracy of these approaches in general.

Whole-genome bisulfite sequencing [6], reduced representative bisulfite sequencing [6b], anchored bisulfite sequencing [7], and enrichment-based [8] methods interrogate 5mC modifications in cfDNA in high-throughput manners. The tumor's tissue-of-origin could be obtained using tissues-specific 5mC markers, and the use of combinations of 5mC-bearing loci may lead to screening of multiple cancer types from one blood sample. Furthermore, targeted deep DNA methylation analysis [9] based on PCR amplification and molecular inversion probes (MIP) [10] could interrogate multiple known sites. However, high sequencing depth and cost, resolution of profiling, complicated probe design, requirements of relatively large amount of input material, and insufficient sensitivity and robustness limit their clinical application using cfDNA.

### SUMMARY OF THE DISCLOSURE

To address the limitations of the techniques available for 5mC detection, the inventors have developed a multiplex 5mC marker barcode counting (MMBC) to quantify 5mC markers in DNA, in which multiple 5mC markers could be targeted for capture and linear amplification. The inventors' method provides highly sensitive and specific quantification of multiple 5mC loci. Accordingly, aspects of the disclosure relate to a method for detecting methylated or unmethylated cytosines in one or more regions of target nucleic acids, the method comprising i) combining a solution comprising the target nucleic acids with a deaminating agent to convert unmethylated cytosines in the target nucleic acids to uracils; ii) next contacting the solution with at least two probes under conditions that allow for the hybridization of the two probes to one target nucleic acid region; wherein a terminal end from each probe hybridizes adjacently to the target nucleic acid region; iii) contacting the solution comprising the hybridized probes and target nucleic acids with a ligase under conditions that allow for the ligation of the terminal ends of the adjacently hybridized probes; and iv) detecting the adjacently hybridized ligated probes in the solution.

Further aspects relate to a method for detecting methylated or unmethylated cytosines in one or more regions of target nucleic acids, the method comprising i) deaminating the target nucleic acids to convert unmethylated cytosines to uracils; ii) hybridizing at least two probes to one target nucleic acid region; wherein a terminal end from each probe hybridizes adjacently to the target nucleic acid region; iii) ligating the terminal ends of the adjacently hybridized probes; and iv) detecting the adjacently hybridized ligated probes in the solution.

Further aspects relate to a kit comprising at least one probe that hybridizes to a target region, wherein the probe hybridizes to fully methylated CpG target region that has been deaminated. In some embodiments, the kit comprises at least two probes. In some embodiments, the kit further comprises one or more of a deaminating agent, ligase, polymerase, and exonuclease. In some embodiments, the kit comprises reagents for isolating target nucleic acids from a biological sample, for amplifying nucleic acids from a sample by PCR, and/or for amplifying nucleic acids by real-time or quantitative.

Further aspects relate to a nucleic acid probe comprising a UID and a hybridization region that hybridizes to fully methylated DNA of a target DNA. In some embodiments, the probe comprises DNA. In some embodiments, the probe hybridizes to a hypermethylation-associated disease region. The term “hypermethylation-associated disease region” refers to a region that, in its state of CpG hypermethylation, is known to be a biomarker for a disease or condition, such as cancer.

In some embodiments, the methods of the disclosure are for diagnosing a disease or condition based on the detection of hypermethylation in target regions. In some embodiments, the disease or condition is cancer. In some embodiments, the methods of the disclosure are for detecting hypermethylation in a target DNA region in a subject.

In some embodiments, the deaminating agent comprises bisulfite. It is contemplated that other deaminating agents may be used in the methods of the disclosure, such as deaminases (e.g. cytidine deaminase or activation-induced cytidine deaminase), which are enzymes that catalyze deamination. A deaminating agent is one that deaminates unmethylated cytosine residues to uracil, leaving 5′-methylcytosine (5-mC) or 5′ hydroxymethyl cytosine (5-hmC) intact. Comparison of sequence information between the reference genome and bisulfite-treated DNA can provide information about cytosine methylation patterns. Embodiments include methods in which one or more steps include incubating a substrate with a deaminating agent under conditions to promote deamination of the substrate by the deaminating agent. Embodiments include where substrates are deaminated by at least about 50, 55, 60, 65, 70, 75, 80, 85, 90, 95, 96, 97, 98, 99, 99.5% or more (or any range derivable therein).

In some embodiments, at least one of the probes comprise a unique identifier (UID). The UID can be a polynucleotide of at least, at most, or exactly 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200 or more (or any range derivable therein) nucleotides in length. In some embodiments, the UID is specific for the target nucleic acid region. In some embodiments, the UID is on the tail of the probe. The inclusion of UIDs may be to facilitate the determination of the presence of hybridization of the probe and a target region, which may be an indication of hypermethylation at the target region when the probe is designed to bind to hypermethylated or fully methylated target regions. Each UID may be unique to a specific target region. The unique portions of the UIDs may be continuous along the length of the probe or the UID may include stretches of nucleic acid sequence that is not unique to any one barcode. It is contemplated that a probe may contain more than one UID, such as a UID indicating a specific target region, and another UID indicating a specific tissue type, cell type, assay condition, disease state, etc. . . . .

In some embodiments, the method comprises repeating steps ii and/or iii more than one time. In some embodiments, steps ii) (hybridization) and iii) (ligation) are repeated at least 1, 2, 3, 4, 5, 6, 7, 8, 9, 10, 11, 12, 13, 14, 15, 16, 17, 18, 19, 20, 21, 22, 23, 24, 25 26, 27, 28, 29, 30, 31 32, 33, 34, 35, 36, 37, 38, 39, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 (or any derivable range therein) times. For example, embodiments of the methods may comprise the performance of steps i, ii iii ii iii and iv in sequential order when step ii and iii is repeated once. In some embodiments, the methods comprise the performance of the steps in the following order: i, (ii, iii)ₙ, and iv, wherein n is 1, 2, 3, 4, 5, 6, 7, 8, 9, 10, 11, 12, 13, 14, 15, 16, 17, 18, 19, 20, 21, 22, 23, 24, 25 26, 27, 28, 29, 30, 31 32, 33, 34, 35, 36, 37, 38, 39, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 (or any derivable range therein). In some embodiments, ii and/or iii is repeated prior to the detection step iv. In some embodiments, repeating steps ii and iii comprises thermal cycle processing that repeats hybridization and ligation.

In some embodiments, the methods further comprise a denaturing step. In some embodiments, the denaturing step precedes hybridization. In some embodiments, step ii comprises: iia) incubating the solution under conditions sufficient for the denaturation of nucleic acids and iib) next contacting the solution with at least two probes under conditions that allow for the hybridization of the two probes to one target nucleic acid region; wherein a terminal end from each probe hybridizes adjacently to the target nucleic acid region. In some embodiments, the denaturation of nucleic acids comprises the dissociation of double stranded nucleic acid into single stranded nucleic acids. In some embodiments, the denaturation step comprises heating the sample to at least, at most, or exactly 80, 85, 90, 95, 100, 105, 110, 115, 120, 125, 130, 135, or 150° C. (or any derivable range therein). In some embodiments, the hybridization step comprises heating the sample to at least, at most, or exactly 15, 20, 25, 30, 35, 40, 45, 50, 55, 60, 65, 70, 75, or 80° C. (or any derivable range therein). In some embodiments, the ligation step iii) comprises heating the sample to at least, at most, or exactly 15, 20, 25, 30, 35, 40, 45, 50, 55, 60, 65, 70, 75, or 80° C. (or any derivable range therein). In some embodiments, cycles of steps ii and iii are performed by denaturing at 85-105° C. and hybridizing and/or ligating the DNA by incubating at 40-70° C.

In embodiments, which involve a control, such embodiments, may further comprise repeating steps ii and/or iii more than one time and or involve a denaturing step as described above.

In some embodiments, detecting the adjacently hybridized ligated probes in the solution comprises determining the identity of and/or quantitating the number of each different UID(s) in the ligated probe. The identity and quantity of the number of detected UIDs is directly relatable to the number of target regions hybridized by the probe. Therefore, detection of a UID can be equated to hypermethylation of a target region when the probe hybridizes to hypermethylated DNA of the target region. The UIDs and/or associated ligated probes may be quantified or determined by methods known in the art, including quantitative sequencing (e.g., using an Illumina® sequencer) or quantitative hybridization techniques (e.g., microarray hybridization technology or using a Luminex® bead system). Sequencing methods are further described herein.

In some embodiments, one or both of the probes comprise a primer binding site. In some embodiments, both probes comprise a primer binding site. In some embodiments, one or both probes comprise 1, 2, or 3 different primer binding sites. One or more of the primer binding sites may be uniform across multiple probes during multiplexing applications. In some embodiments, the primer binding sites are at least, at most, or exactly 8, 9, 10, 11, 12, 13, 14, 15, 16, 17, 18, 19, 20, 21, 22, 23, 24, or 25 (or any derivable range therein) nucleotides in length. In some embodiments, the primer binding site is on the tail of the probe.

In some embodiments, detection of the adjacently hybridized ligated probes in the solution comprises linear detection. In some embodiments, the method further comprises PCR amplification of the adjacently hybridized ligated probes. In some embodiments, the method further comprises real-time PCR amplification of the adjacently hybridized ligated probes. In some embodiments, the method further comprises quantitative PCR amplification of the adjacently hybridized ligated probes. In some embodiments, detecting the adjacently hybridized ligated probes comprises quantitative PCR. In some embodiments, detecting the ligated adjacently hybridized probes comprises sequencing the probes. In some embodiments, detecting the adjacently hybridized probes comprises one or more sequencing methods described herein.

In some embodiments, the method further comprises ligation of one or more adaptors to the adjacently hybridized ligated probes. In some embodiments, at least two target regions are detected. In some embodiments, at least 2, 3, 4, 5, 6, 7, 8, 9, 10, 12, 15, 20, 25, 30, 35, 40, 45, 50, 75, 100, 125, 150, 200, 225, 250, 275, 300, 350, 400, 500, 600, 700, 800, 900, 1000, 1500, 2000, or 3000 (or any derivable range therein) target regions are detected. In some embodiments, the at least two target regions are detected from target nucleic acids in the same solution. In some embodiments, at least 2, 3, 4, 5, 6, 7, 8, 9, 10, 12, 15, 20, 25, 30, 35, 40, 45, 50, 75, 100, 125, 150, 200, 225, 250, 275, 300, 350, 400, 500, 600, 700, 800, 900, 1000, 1500, 2000, or 3000 (or any derivable range therein) target regions are detected from target nucleic acids in the same solution.

In some embodiments, ii) comprises contacting the solution with at least four probes under conditions that allow for the hybridization of the two probes to one target nucleic acid region and two probes to a different or second target nucleic acid region. In some embodiments, ii) comprises contacting the solution with at least 2 times n probes under conditions that allow for the hybridization of two probes to one target nucleic acid region and two probes to a different or second target nucleic acid region, and two probes to a nth target region, wherein n=3, 4, 5, 6, 7, 8, 9, 10, 12, 15, 20, 25, 30, 35, 40, 45, 50, 75, 100, 125, 150, 200, 225, 250, 275, 300, 350, 400, 500, 600, 700, 800, 900, 1000, 1500, 2000, or 3000. Therefore, the number of probes used is 2 times the number of target regions (n), and each set of 2 probes hybridizes adjacently to one target region or to one target region state (such as fully CpG methylated, unmethylated, or partially CpG methylated).

In some embodiments, the target nucleic acid region comprises at least 40 contiguous nucleic acids. In some embodiments, the target nucleic acid region comprises at least, at most, or exactly 10, 15, 20, 25, 30, 35, 40, 45, 50, 55, or 60 nucleic acids. In some embodiments, at least, at most, or exactly 5, 10, 15, 20, 25, 30, 35, 40, 45, 50, 55, or 60 nucleic acids in the probe are capable of hybridizing to a target nucleic acid region or target nucleic acid region state.

In some embodiments, one or more of the probes comprises a 3′ or 5′ tail. In some embodiments, the tail is at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 (or any derivable range therein) nucleotides in length. In some embodiments, the tail comprises phosphorothioate-modified nucleic acids. In some embodiments, the tail comprises at least 2 phosphorothioate-modified nucleic acids. In some embodiments, the tail comprises four phosphorothioate-modified nucleic acids. In some embodiments, the tail comprises at least, at most, or exactly 1, 2, 3, 4, 5, 6, 7, 8, 9, 10, 12, 15, 16, 18, 20, 22, 24, 26, 28, or 30 phosphorothioate-modified nucleic acids. In some embodiments, the entire probe comprise phosphorothioate-modified nucleic acids. In some embodiments, one or more of the probes comprises a terminal cap. In some embodiments, one or more of the terminal ends of the probe are modified. In some embodiments, the modification comprises a terminal cap.

In some embodiments, the method further comprises contacting the solution comprising the ligated probes with an exonuclease. Exonucleases are known in the art and include, for example, lambda exonuclease, T7 exonuclease, and T5 exonuclease.

In some embodiments, steps i)-iv) are performed in sequential order.

In some embodiments, the probe hybridizes to fully methylated CpG target nucleic acids. In some embodiments, the probe hybridizes to fully unmethylated CpG target nucleic acids. In some embodiments, the probe hybridizes to partially methylated CpG target nucleic acids. In some embodiments, the probe specifically hybridizes to fully methylated CpG target nucleic acids that have been deaminated and does not hybridize to fully unmethylated CpG target nucleic acids that have been deaminated. In some embodiments, the probe specifically hybridizes to fully unmethylated CpG target nucleic acids that has been deaminated and does not hybridize to fully methylated CpG target nucleic acids that have been deaminated. In some embodiments, the probe hybridizes to partially methylated CpG target nucleic acids. In some embodiments, the probe hybridizes to a target region in which at least, at most, or exactly 1, 2, 3, 4, 5, 6, 7, 8, 9, 10, 11, 12, 13, 14, 15, 16, 17, 18, 19, or 20 (or any derivable range therein) CpGs out of 1, 2, 3, 4, 5, 6, 7, 8, 9, 10, 11, 12, 13, 14, 15, 16, 17, 18, 19, or 20 CpGs in the target region is methylated.

In some embodiments, the solution comprising the target nucleic acids comprises less than 1 ng of nucleic acids or target nucleic acids. In some embodiments, the solution comprising the target nucleic acids comprises at least or at most 0.1, 0.5, 1, 5, 10, 50, 100, 250, 500, or 750 ng or 1, 2, 4, 8, 10, 15, 20, or 30 micrograms (or any derivable range therein) of nucleic acids or target nucleic acids.

In some embodiments, the method further comprises: i) combining a solution comprising the control nucleic acids with a deaminating agent to convert unmethylated cytosines to uracils; ii) next contacting the solution with at least two control probes under conditions that allow for the hybridization of the two probes to one control nucleic acid region; wherein a terminal end from each probe hybridizes adjacently to the control nucleic acid region; iii) contacting the solution comprising the hybridized control probes and control nucleic acids with a ligase under conditions that allow for the ligation of the terminal ends of the adjacently hybridized control probes; and iv) detecting the adjacently hybridized ligated control probes in the solution.

In some embodiments, the control probes hybridize to unmethylated, deaminated control nucleic acids. In some embodiments, the control probes hybridize to methylated control nucleic acids. In some embodiments, the control probes hybridize to partially methylated nucleic acids. In some embodiments, the control nucleic acids are in the same solution as the target nucleic acids. In some embodiments, the control nucleic acids are in a different solution as the target nucleic acids.

In some embodiments, the control nucleic acids comprise a known percentage of fully methylated control target nucleic acids. In some embodiments, the method further comprises construction of a calibration curve.

In some embodiments, the ligase comprises a thermostable ligase. Ligases useful in the methods and kits of the disclosure are known in the art and include, for example, T4 DNA ligase, SplintR® Ligase (New England BioLabs), T7 DNA ligase, thermostable 5′ App DNA/RNA ligase.

In some embodiments, the method further comprises library construction from the hybridized probes. In some embodiments, a library comprising nucleic acids corresponding to hybridized ligated probes is constructed. Method for library construction are known in the art and include, for example, first strand synthesis, adaptor ligation, plasmid construction, universal primer binding, PCR amplification, and sequencing.

In some embodiments, the target and/or control nucleic acids comprise cell free DNA. In some embodiments, the target and/or control nucleic acids comprise nucleic acids isolated from a biological sample described herein. In some embodiments, the target and/or control nucleic acids are isolated from a cell or population of cells. In some embodiments, the target and/or control nucleic acids are isolated from a serum, urine, stool, cerebrospinal fluid, biopsy, fine needle biopsy, genomic DNA, frozen, or formalin-fixed, paraffin-embedded sample. In some embodiments, the target and/or control nucleic acids are isolated from a sample from a patient with a disease or disorder or from a patient suspected of having a disease or disorder. In some embodiments, the disease or disorder comprises cancer or autoimmunity.

In some embodiments, the number of CpGs in each target region is 5-12. In some embodiments, the number of CpGs in ease target region is 6-12, 7-12, 8-12, or 9-12. In some embodiments, the number of CpGs in ease target region is at least, at most, or exactly 1, 2, 3, 4, 5, 6, 7, 8, 9, 10, 11, 12, 13, 14, 15, 16, 17, 18, 19, or 20 (or any derivable range therein). In some embodiments, the number of CpGs that a probe hybridizes is at least, at most, or exactly 1, 2, 3, 4, 5, 6, 7, 8, 9, 10, 11, 12, 13, 14, 15, 16, 17, 18, 19, or 20 (or any derivable range therein). In some embodiments, the number of CpG hybridized by a probe pair is at least, at most, or exactly 1, 2, 3, 4, 5, 6, 7, 8, 9, 10, 11, 12, 13, 14, 15, 16, 17, 18, 19, or 20 (or any derivable range therein).

In some embodiments, the method further comprises determining the total amount of target region. In some embodiments, determining the total amount of target region comprises performing a non-deamination control sample using target probes that hybridize to undeaminated target region. A non-deamination control may comprise contacting a solution with at least two control probes under conditions that allow for the hybridization of the two probes to one undeaminated target nucleic acid region; wherein a terminal end from each probe hybridizes adjacently to the target nucleic acid region; iii) contacting the solution comprising the hybridized probes and target nucleic acids with a ligase under conditions that allow for the ligation of the terminal ends of the adjacently hybridized probes; and iv) detecting the adjacently hybridized ligated probes in the solution.

The present disclosure relates to the methods and kits for manipulating, producing, creating, amplifying purifying, isolating, analyzing, assaying, measuring, sequencing, and evaluating methylation of nucleic acids. What is provided is a way to amplify DNA from very limited biological samples (e.g. from small number of cells, body fluids, or biopsy samples) with DNA methylation (5-methylcytosine, 5mC) information faithfully copied from the starting material.

Methods involve a polymerase that replicates methylated genomic DNA. Strand displacement polymerase are used in some embodiments. DNA polymerase such phi29 polymerase, Klenow fragment, Bacillus stearothermophilus DNA polymerase (BST), T4 DNA polymerase, T7 DNA polymerase, or DNA polymerase I may be used.

Further aspects relate to a kit comprising compositions of the disclosure and instructions for use.

In some embodiments, methods further comprise testing the patient for an infection, such as a viral infection or diagnosing a patient with an infection, such as a viral infection.

Any embodiment discussed in the context of an antibody may be implemented in any method embodiment discussed herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Depicted is a schematic describing a multiplex 5mC marker barcode counting (MMCB) method to detect 5mC hypermethylation in plasma cell-free DNA. Multiple hypermethylated loci can be linearly amplified simultaneously in one tube, with the use of molecular barcode to further improve quantification accuracy. MMBC-seq was applied for non-invasion distinguishing colorectal cancer patient from healthy control.

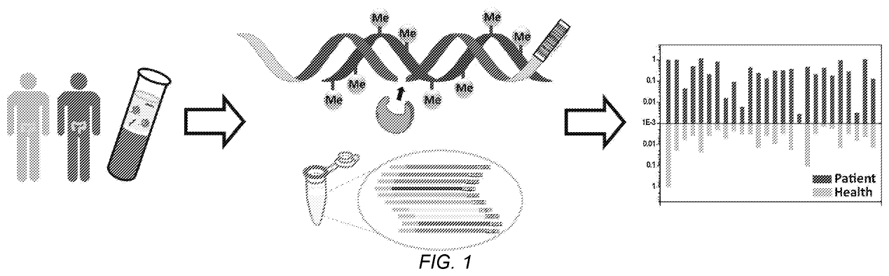

FIG. 2. The strategy of multiplex 5mC marker barcode counting (MMBC). Pairs of probes can recognize the 5mC markers (after bisulfite treatment) and be ligated through thermocycles. 3′ and 5′-ends of forward and reverse probes are protected by phosphorothioate, which resists digestion of exonucleases. The linear, unique identifier (UID)-barcoded DNA, carrying the quantitative information of input DNA material, can be detected by qPCR or NGS platform.

FIG. 3A-E The sensitivity and linear amplification of MMBC: (A) The 60-mer model DNA probes (VIM 5mC model and VIM C model) carrying 10 CpG sites as indicated and their sequences after bisulfite treatment. A pair of VIM probes are shown to interrogate these methylation sites. Ligation occurs only in the presence of correct base pairing. The linear product DNA molecules could be amplified and detected by different readout platforms. (B) The kinetics of thermocycle-based linear amplification, ΔCq vs. N, ΔCq=Cq₍₀₎−Cq₍ₙ₎. Insert: ΔCq vs. Lg n. In the inventors system, VIM 5mC model showed significant linear amplification as compared to unmethylated VIM model. (C) Quantification performance of MMBC on VIM models: the fluorescence curves with 6, 6*10¹, 6*10², 6*10³, 6*10⁴, 6*10⁵ copies of VIM 5mC model and 6*10⁵ and 6*10⁶ copies of VIM C model. (D) Multiple loci detection: the linear calibration curves of BMP3, NGRG4, TFPI2, VIM, ACTB sites in 37.5 pg, 78 pg, 312.5 pg, 1.25 ng and 5 ng fully methylated human genomic DNA (FMG). (E) The spike-in test of the above five loci by using 100 pg, 1 ng and 10 ng FMG in 10 ng unmethylated human genomic DNA (UMG). Error bars indicate mean±SD, n=3.

FIG. 4A-C MMBC-seq of colorectal cancer (CRC) multiple loci in FMG: (A) Comparison of 10 ng FMG and 10 ng UMG. UID counts of 26 sites were normalized by using ACTB. (p=0.0007). (B) The Pearson correlation of loge transformed UID counts between technique replicates of 10 ng, 1 ng and 100 pg FMG. Each dot represents a 5mC locus. (C) The UID counts of corresponding loci detected in 10 ng, 1 ng and 100 pg FMG were shown in histogram with technical replicates.

FIG. 5A-C Spike-in test of MMBC-seq (A-B): (A) Scatterplots showing the correlation of 10 ng, 1 ng and 100 pg FMG spiked into 15 ng of UMG to the libraries constructed by the same concentrations of FMG alone, respectively. (B) The average UID counts of ALX_776, NGFR_557, SEPT9_F1442 and TMEFF2_708, respectively against the varying spike-in amounts of FMG to 15 ng of UMG (red bars). UID count of each locus is the mean of replicates. The MMBC-seq detection of clinical sample (C): colorectal cancer patient and healthy donor were discriminated by using cfDNA in plasma. UID counts of 26 sites were normalized by ACTB. (p<0.0001)

FIG. 6 The amplification folds of VIM 5mC model along the increasing thermal cycle n. The folds showed a positive correlation with the cycle number. Error bars indicate mean±SD, n=3.

FIG. 7 The comparison of performance of Hifi Taq Ligase and Ampligase in the MMBC system. Experiment conditions: 1 μL 5mC VIM model (10 pM), 1 μL C VIM model (10 nM) or no model template was mixed with 1 μL VIM forward/reverse probe (1 μM), 1 μL thermostable ligase and 1 μL corresponding 10× buffer to obtain a 10 μL reaction system. The reaction was subjected to 180 thermocycles, and then analyzed by qPCR quantification. Hifi Taq Ligase showed a slight better performance to distinguish VIM 5mC and C model. In this work, Hifi Taq Ligase was selected to carry out all MMBC experiments.

FIG. 8A-B System optimization using a test panel with 100 pairs of probes: (A) temperature optimization, performed by 100% mC, 10% mC and C standards. The ratios 100% mC signal/C signal and 10% mC signal/C signal of each sites were plotted. (B) the comparison of optimized probes and all probes by target length, GC % and Tm. P1 and P2 indicate the two side of a pairs of probe.

FIG. 9 The size distribution of a typical MMBC-seq library. The 35 bp and 10380 bp markers were loaded together with library sample. FU, fluorescence units.

FIG. 10 Comparison of quantification by UID counts and reads. The numbers of detected TMEFF2_245 and ALX4-112 were plotted against the amount of input FMG at 100 pg, 1 ng and 10 ng. UID quantification showed better linear correlation.

### DESCRIPTION OF ILLUSTRATIVE EMBODIMENTS

The detection of DNA methylation markers in biological fluids presents a promising non-invasion approach for early diagnosis and monitoring of human diseases. A multiplex, site-specific, and cost-effective assay to detect multiple 5mC markers in plasma is currently lacking. Here the inventors present a multiplex 5mC marker barcode counting (MMBC) method to detect 5mC hypermethylation in cell-free DNA (cfDNA). Multiple hypermethylated loci can be linearly amplified simultaneously in one tube, with the use of unique identifier (UID) molecular barcodes to further eliminate bias and duplicates. The inventors confirmed that the MMBC-based qPCR approach can sensitively amplify and quantify five hypermethylated loci using as low as 13 copies of the haploid genome input material. MMBC-seq was applied to target a series of hypermethylated loci associated with colorectal cancer (CRC) in plasma cfDNA, showing excellent performance in distinguishing CRC patient from healthy control. The robust and straightforward method targeting multiple loci provides high accuracy and flexible convenient for clinical applications.

### I. DEFINITIONS

A “CpG island” or simply “CpG” as used herein refers to regions of DNA with a high G/C content and a high frequency of CpG dinucleotides relative to the whole genome of an organism of interest. Also used interchangeably in the art is the term “CG island.” The ‘p’ in “CpG island” refers to the phosphodiester bond between the cytosine and guanine nucleotides.

A methylated cytosine refers to a DNA base with the following structure:

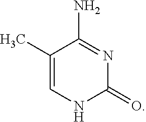

The term “fully methylated region” is meant to encompass a region in which each cytosine of each CpG is methylated.

The term terminal end refers to a 3′ or 5′ terminal nucleotide of a nucleic acid. The 3′ terminal end may comprise a hydroxyl group, and the 5′ terminal end may comprise a phosphate. In some embodiments, the 3′ terminal end is modified. In some embodiments, the 3′ terminal end is modified with a cap, such as a 3′-phosphate to prevent polymerase non-specific extension. Other modifications include a 3′ dideoxy Cytosine, a C3 spacer at the 3′ or 5′ end. For example, the C3 Spacer phosphoramidite can be incorporated at the 5′-end of the probe or multiple C3 spacers can be added at either end of an oligo to introduce a long hydrophilic spacer arm that may be used for the attachment of fluorophores or other pendent groups or to block non-specific extension. In some embodiments, the terminal end may be modified with an amino, such as a 3′ amino to block non-specific primer extension.

The term “unique identifier” or “UID” refers to a nucleic acid sequence that can be used to quantitate or identify a target region that it is associated with.

The term “tail” with respect to a probe or primer refers to a region that does not hybridize to a target nucleic acid region, whether the region is hypermethylated, fully methylated, or unmethylated. The inclusion of a tail on a probe or primer may be used to introduce additional nucleic acid regions, such as a UID, a primer binding site, or a universal primer binding site. Inclusion of these sequences on the tail can facilitate PCR amplification and/or sequencing of the ligated probes.

The term “target nucleic acids” refers to nucleic acids for which analysis is sought. The nucleic acids may be RNA or DNA and may be fragmented, genomic, cell-free, etc. . . . .

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989; Wetmur, 1991; and Owczarzy et al., 2008, which are incorporated herein by reference). Thus the design of appropriate primers and probes, and the conditions under which they hybridize to their respective targets is well within the routine skill of the person skilled in the art.

The term “adjacently hybridized,” “hybridizes adjacently,” or the like refers to the hybridization of the probes to a contiguous nucleic acid such that the terminal ends hybridize to adjacent nucleotides (no intervening nucleotides) of the target region.

A phosphorothioate-modified nucleotide refers to where a phosphorothioate (PS) bond substitutes a sulfur atom for a non-bridging oxygen in the phosphate backbone of an oligo. This modification renders the internucleotide linkage resistant to nuclease degradation. Phosphorothioate bonds can be introduced between the last 3-5 nucleotides at the 5′- or 3′-end of the probe to inhibit exonuclease degradation. Including phosphorothioate bonds throughout the entire oligo will help reduce attack by endonucleases as well.

The term “calibration curve” is a general method for quantitating a substance in an unknown sample by comparing the unknown to a set of standard samples of a known amount, such as a known percentage or fraction of fully methylated, partially methylated, or unmethylated target region.

The term “library” refers to a collection (e.g., to a plurality) of vehicles that comprise the amplified genomic methylated DNA molecules. The vehicle may be a vector, construct, array, or other physical vehicle. A “vector” or “construct” (sometimes referred to as gene delivery or gene transfer “vehicle”) refers to a macromolecule, complex of molecules, or viral particle, comprising a polynucleotide to be delivered to a host cell, either in vitro or in vivo. The polynucleotide can be a linear or a circular molecule. One of skill in the art would be well equipped to construct a vector through standard recombinant techniques (see, for example, Maniatis et al., 1988 and Ausubel et al., 1994, both incorporated herein by reference). An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

### II. ASSAY METHODS

A. Ligation Methods

Aspects of the disclosure include the ligation of nucleic acids. It is contemplated that any suitable ligase may be used and easily selected by one skilled in the art. Exemplary ligases include E. coli DNA ligase, T4 DNA ligase, mammalian ligases, and thermostable ligases. Embodiments of the disclosure may also include incubation of one or more assay components with a phosphatase. For example, embodiments include incubation of one or more probes, target nucleic acids, controls, hybridized probes, and/or amplified hybridized probes with a phosphatase. The incubation may be a pre-incubation, meaning that it takes place prior to contact with the composition.

B. Sequencing

Aspects of the disclosure may include sequencing nucleic acids to detect hybridization of the probes and/or to determine the identity of the UID. Described below are exemplary methods for performing such sequencing reactions.

1. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

2. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform, which is now owned by Life Technologies.

3. 454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

4. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massivelly parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

5. Solid Sequencing.

Applied Biosystems' (now a Thermo Fisher Scientific brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

6. Ion Torrent Semiconductor Sequencing.

Ion Torrent Systems Inc. (now owned by Thermo Fisher Scientific) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

7. DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

8. Heliscope single molecule sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

9. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.]

C. Nucleic Acid Assays

Aspects of the methods include assaying nucleic acids to determine methylation status of CpG regions of DNA. In some embodiments, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze nucleic acids. Such assays include, but are not limited to, nucleic amplification, polymerase chain reaction, quantitative PCR, RT-PCR, in situ hybridization, digital PCR, dd PCR (digital droplet PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a barcode region, probe, or oligo described herein. The term “primer” or “probe” as used herein, is meant to encompass any nucleic acid that is capable of priming the synthesis of a nascent nucleic acid in a template-dependent process and/or pairing with a single strand of an oligo of the disclosure, or portion thereof. Typically, primers are oligonucleotides from ten to twenty and/or thirty nucleic acids in length, but longer sequences can be employed. Primers may be provided in double-stranded and/or single-stranded form, although the single-stranded form is preferred.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In one embodiment, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one embodiment, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable DNA fragment that is similar or larger than the target DNA fragment and in which the abundance of the DNA representing the internal standard is roughly 5-100 fold higher than the DNA representing the target nucleic acid region.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target DNA fragment. In addition, the nucleic acids isolated from the various samples can be normalized for equal concentrations of amplifiable DNAs.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be 50, 100, 200, 300, 400, 500 or more probes/cm2.

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain embodiments may involve the use of arrays or data generated from an array. Data may be readily available. Moreover, an array may be prepared in order to generate data that may then be used in correlation studies.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

### III. BIOLOGICAL SAMPLES

It is generally desirable to be able sensitively, specifically, qualitatively and/or quantitatively to detect methylated DNA in a sample, including for example in fixed or fresh cells or tissues or in a cell free biological sample. It may be particularly desirable to detect, sequence, or evaluate methylated DNA in a single cell. For example, in population-based assays that analyze the content of many cells, molecules in rare cells may escape detection due to the low abundance of material to evaluate. This is similarly true for cell-free biological samples.

The sample may, for example, be derived from a tissue or organ of the body, or from a bodily fluid. Such a sample will advantageously be or comprise a cell or group of cells such as a tissue. The sample may, for example, be a colon, lung, pancreas, prostate, skin, thyroid, liver, ovary, endometrium, kidney, brain, testis, lymphatic fluid, blood, plasma, urinary bladder, or breast sample, or comprise colon, lung, pancreas, prostate, skin, thyroid, liver, ovary, endometrium, kidney, brain, testis, lymphatic fluid, blood, urinary bladder, or breast cells, groups of cells or tissue portions. Samples may be cultured or harvested or biopsied cell or tissue samples, e.g. as mentioned above, in which the methylated genomic DNA may be detected to reveal the qualitative or quantitative nature of the methylation that it is present, or the nucleotide sequence of methylated nucleic acids at one or more specific genes, regions, CpG islands and the like. The sample of cells may be freshly prepared or may be prior-treated in any convenient way such as by fixation or freezing. Accordingly, fresh, frozen or fixed cells or tissues may be used, e.g. FFPE tissue (Formalin Fixed Paraffin Embedded). Thus, tissue sections, treated or untreated, may be used.

DNA may be isolated from an organism of interest, including, but not limited to eukaryotic organisms and prokaryotic organisms, preferably mammalian organisms, such as humans.

### IV. KITS

The disclosure additionally provides kits for modifying cytosine bases of nucleic acids and/or subjecting such modified nucleic acids to further analysis. The contents of a kit can include one or more of the following reagents described throughout the disclosure such as deaminating reagents, such as bisulfite, probes, including modified probes, such as thiophosphorate-modified probes, primers, reagents for performing primer extension and PCR, such as a polymerase, buffers, and nucleotides, sequencing reagents, and sequencing primers.

Each kit may include any components that are useful for amplifying the nucleic acid, or sequencing the nucleic acid, or other applications of the present disclosure as described herein. The kit may optionally provide additional components that are useful in the procedure. These optional components include buffers, capture reagents, developing reagents, labels, reacting surfaces, means for detection, control samples, instructions, and interpretive information.

### V. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Multiplex 5mC Marker Barcode Counting for Methylation Detection in Cell-Free DNA

A. Results

The methods of the disclosure provide for highly sensitive (LOD 6 copies for single model site, and ˜13 copies for multiple markers) and specific quantification (10-5) of multiple 5mC loci. The inventors show that MMBC-seq targeting 25 5mC loci associated with colorectal cancer (CRC) successfully distinguishes CRC patient and health control using plasma cfDNA. Using the next generation sequencing (NGS) platform, MMBC-seq can detect multiple loci with high accuracy and low cost using trace amounts of cfDNA in plasma.

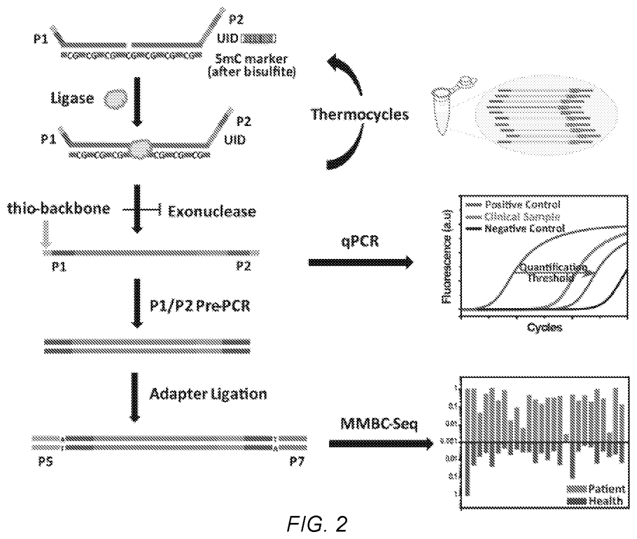

As shown in FIG. 2, a pair of probes can be designed for each targeted region covering 40-80 bp with multiple mCpG. The split site is located in the middle of mCpG. Each probe possesses an overhang universal primer region (P1/P2) for the first round of PCR amplification. An 8-12 nt random barcode is inserted as unique identifier (UID)[11]. 3′ and 5′-ends of the overhangs are protected by phosphorothioate and terminal capping modifications. After bisulfite treatment, the target DNA and the matched probe pair can be annealed. Thermostable ligase can fill the gap between the forward probe and the reverse probe to form a linear DNA. Importantly, the inventors employed the typical PCR thermal cycles which mediate the hybridization and ligation events in MMBC. Under such conditions a linear amplification of hybridization and ligation was achieved, which eliminates bias typically associated with and seriously accumulated in exponential amplification approaches. In addition, taking advantage of the unique linear capture and amplification, UID molecular barcode was added to each ligated DNA molecule in order to further digitally improve the quantification accuracy by barcode counting, as well as identifying and removing duplicates and bias during subsequent amplification and sequencing. In addition, the inventors employed exonucleases to digest the non-ligated probes and the input DNA templates; the ligated linear DNA molecules were protected by the phosphorothioate at both 5′ and 3′ ends. After exonuclease treatment, the UID-barcoded DNA amplicons carrying the digital information of input DNA material can be subjected to NGS or qPCR readout.

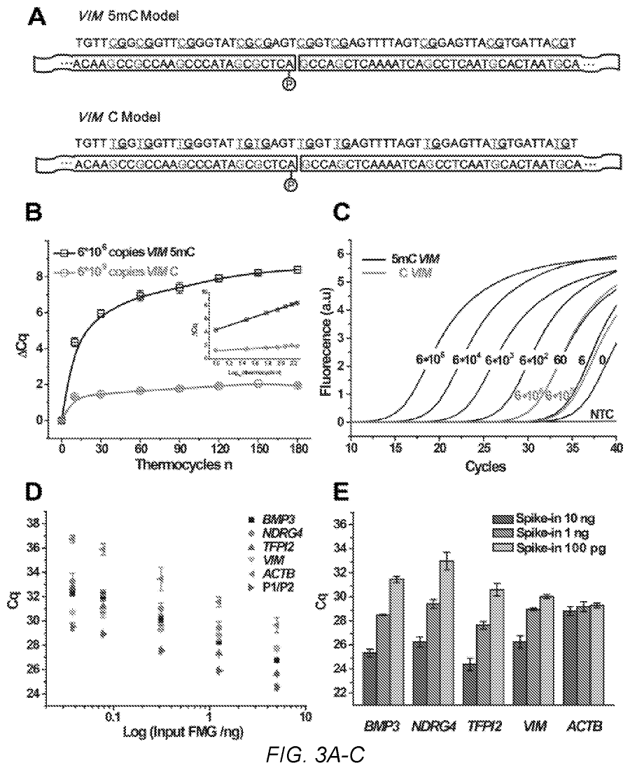

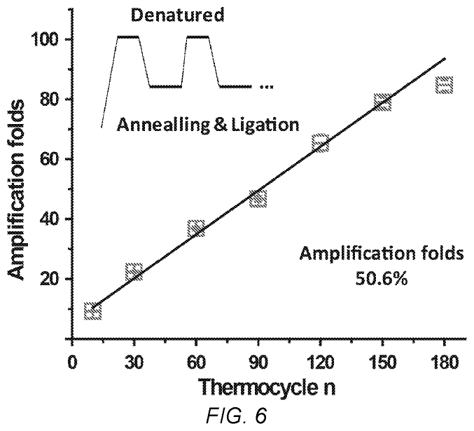

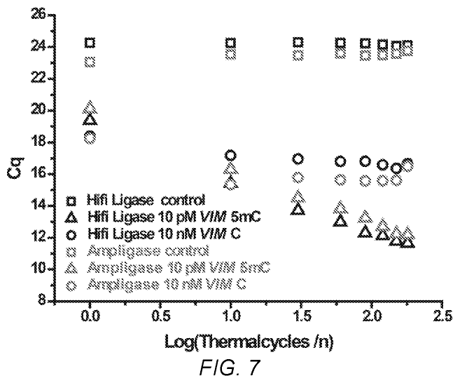

The inventors first confirmed the desired linear amplification by using synthesized VIM model (FIG. 3A), which is a well-known CRC hypermethylation locus.[12] The kinetics of the thermocycle reaction was investigated by performing different rounds of thermocycle in the hybridization step. The ligated product was quantified by qPCR (FIG. 3B). A linear relation between ΔCq and lg (thermocycle number n) was observed: ΔCq=3.24 lg(n)+1.14, R²=0.998 when 6*10⁶ copies of methylated VIM 5mC model were used. The result indicated that 5mC markers on the target DNA were linearly amplified during the thermocycle. The inventors calculated the amplification folds of VIM 5mC versus thermocycle rounds (FIG. 6, folds=0.506n+1, R²=0.969). The average capture and hybridization efficiency per cycle is ˜50.6%, which is much higher than that of other common hybridization-based methods. The inventors also tested this system by using one-thousand-fold unmethylated VIM C control. The ΔCq value of the unmethylated VIM C kept constant after 10 cycles, and its augmentation was significant less than that of 1/1,000 copies of 5mC-methylated VIM (FIG. 3B). The specificity to distinguish VIM with 5mC and unmethylated VIM could be further improved by enduring more rounds of thermocycle. The inventors compared the performance of two different thermostable ligase, Hifi Taq ligase and Ampligase (FIG. 7), and the former one was selected with better performance in the inventors system. Under the experimental conditions, MMBC can linearly quantify 6 to 6*10⁵ copies of methylated VIM, Cq=−3.62 lg (VIM 5mC)+35.6, R²=0.999 (FIG. 3C). It possesses superior specificity (10⁻⁵) to discriminate unmethylated and methylated VIM templates.

Having validated the linear amplification and specific quantification of a single VIM model template, the inventors next investigated if the method can be adapted for multiplex 5mC-methylated DNA templates which could be simultaneously amplified in one reaction. The inventors picked four CRC hypermethylation DNA markers, BMP3, NGRG4, VIM, and TFPI2, and one internal reference ACTB,[13] and detected the five loci by using gradually diluted fully-methylated human genomic DNA (FMG). All five loci were detectable in as low as 37.5 pg FMG input (equivalent to ˜13 copies haploid genome or ˜7 cells). Linear calibration curves of each locus were clearly shown in FIG. 3D. P1 and P2 were located at each end of paired probes, and used to amplify all ligated linear DNA molecules in MMBC. The corresponding linear correlation was R²=0.989 for BMP3, 0.997 for NGRG4, 0.996 for VIM, 0.994 for TFPI2, 0.998 for ACTB, and 0.997 for P1/P2 primers. Although certain qPCR assays claimed to be able to detect less than 10 copies of target DNA, none of these assays could simultaneously amplify five or more loci in one reaction. The input DNA materials have to be split into multiple wells in previous assays for multiplex detections leading to lower sensitivity. MMBC can achieve highly sensitive detection of multiple loci in one reaction as demonstrated.

After confirming the high sensitivity and linear amplification of multiple 5mC-hypermethylated loci, the inventors proceeded to test if they can specifically detect a spiked methylated locus in a mixture of methylated and unmethylated genomic DNA. In this spike-in experiment, 100 pg, 1 ng and 10 ng FMG were mixed with 10 ng unmethylated human genomic DNA (UMG). The results showed that the amplicon number of the housekeeping gene ACTB was steady in all experiments; in contrast, the four 5mC-modified DNA showed gradient increase along with the increased spike-in proportions of FMG (FIG. 3E). Thus, the inventors strategy can capture, amplify, and detect multiple hypermethylated DNA markers existing in low copy numbers within the complex background of human genomic DNA.

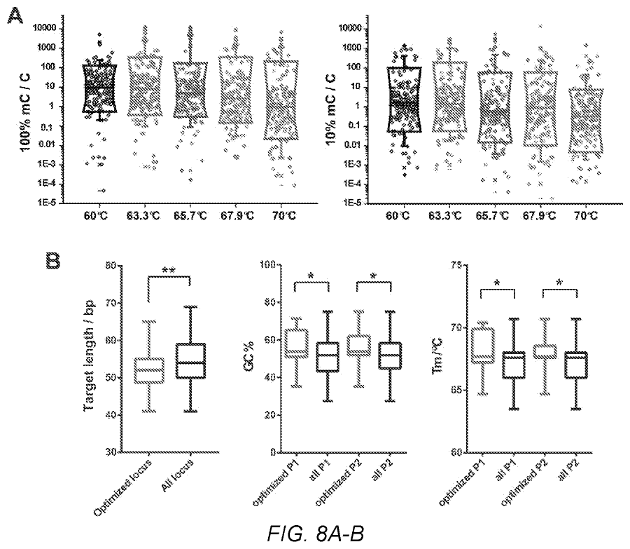

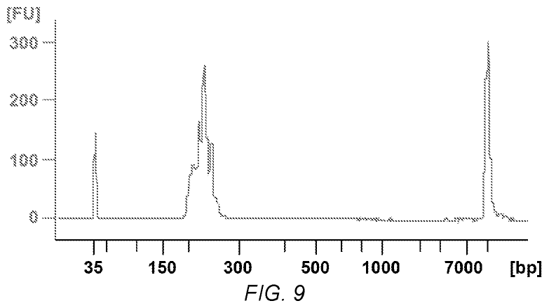

With the high-performance method in hand, the inventors can apply MMBC-seq to circulating tumor DNA in plasma. Before cfDNA panel design, the inventors optimized the multiple system for larger numbers of probes, including reaction temperature of thermocycle and probe design preferences in length, GC content and melting temperature Tm (FIG. 8). Then the inventors designed a series of probes to target four CRC specific hypermethylation genes, SEPT9, TMEFF2, NGFR, ALX4.[14] 25 pairs of MMBC probes were designed to cover the hypermethylation regions of the promoters and exons. A pair of ACTB probe was added as internal standard. MMBC was performed and the resulting products were submitted to library construction and next generation sequencing (FIG. 9, a typical MMBC library). To correct the PCR bias and optical error during library construction and sequencing, raw data were mapped to the targeted regions, in which the MMBC products were counted by UID.

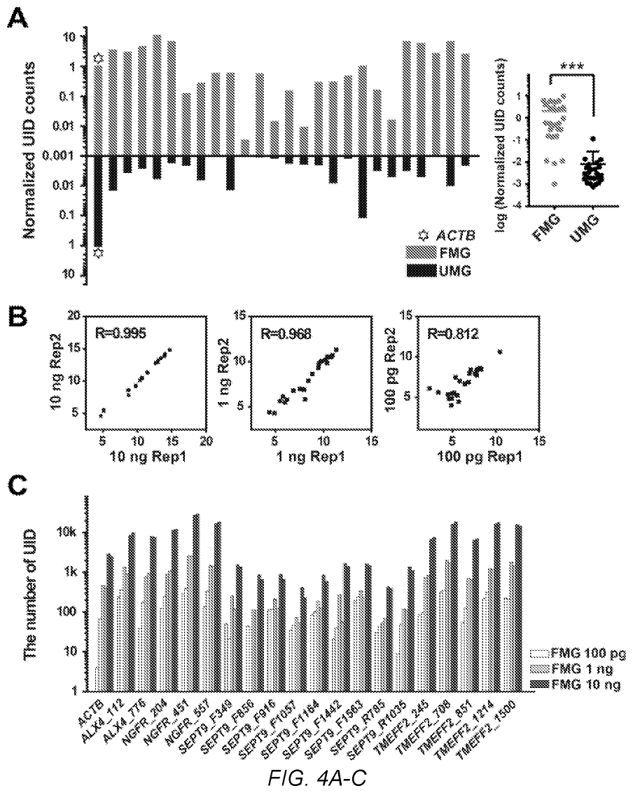

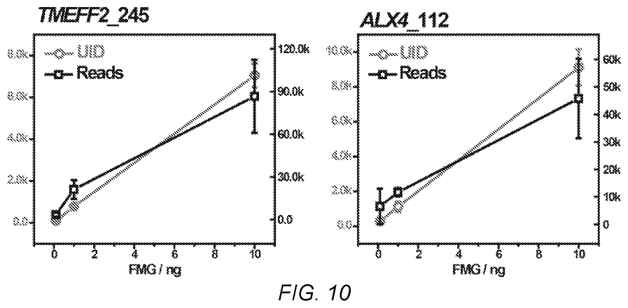

When 10 ng FMG was used as input, 26 sites (26/26) were covered by at least 4 UID counts or 73 reads. The UID counts for each site were normalized using ACTB (FIG. 4A). All detected sites in FMG were 101-103 folds higher than that in UMG. The varied fold changes across different sites are not surprising as the overall efficiency of different sites could be affected by diverse hybridization efficiencies resulted from factors such as GC content, secondary structure, lengths of probes, non-uniform methylation of FMG standard, and spontaneous background ligation. Importantly, high reproducibility between technical replicates was observed for each site (FIG. 4B, R=0.995 for 10 ng FMG, 0.968 for 1 ng FMG, and 0.812 for 100 pg FMG). The counted UID number for each site was shown to be positively correlated to the corresponding hypermethylation locus copy number presented in gDNA input. A linear correlation was obtained among most targeted sites (FIG. 4C). The inventors calculated the on-target efficiency to validate the probe design. Even with the use of 100 pg FMG, the detectable probe ratio was as high as 77%. UID counts also effectively corrected the quantitative linear relationship (e.g. TMEFF2_245 and ALX4 112 in FIG. 10).

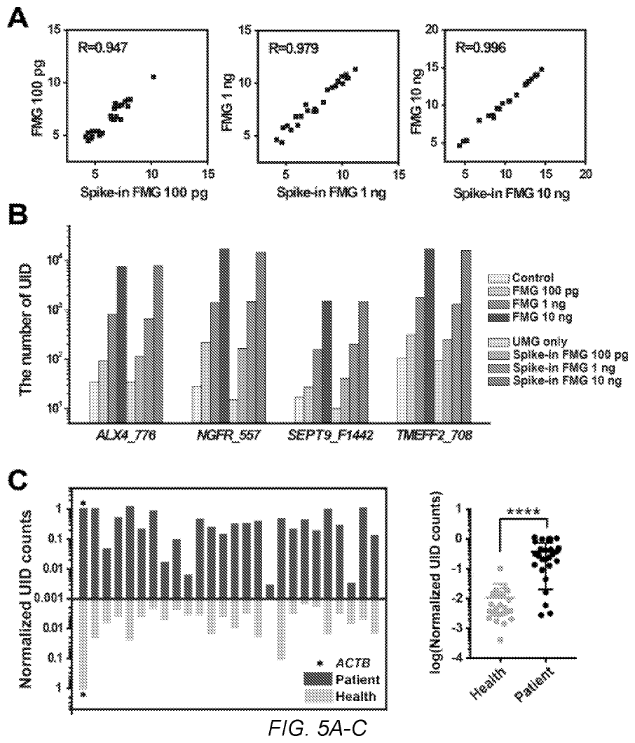

Because circulating tumor DNAs only constitute a small proportion of the total cfDNA, to evaluate the specificity of MMBC in detecting hypermethylated CpG markers in cfDNA, the inventors spiked in 100 pg, 1 ng, and 10 ng FMG into 15 ng UMG. Again, FMG in the FMG-UMG mixture showed high correlation with libraries constructed by FMG alone (FIG. 5A, spike-in 10 ng R=0.996, spike-in 1 ng R=0.979, and spike-in 100 pg R=0.947) and good linearity of each locus (e.g. ALX4-776, NGFR-557, SEPT9-F1442 and TMEFF2-708 illustrated in FIG. 5B). As low as 0.6% hypermethylation marker DNA could be detected accurately.

Lastly, the inventors applied the method to discriminate CRC patient and healthy donor using cfDNA in plasma. The normalized counts of CRC patient in these sites were significant higher than those in healthy donor (FIG. 5C, P<0.0001), confirming that MMBC-seq has great potential to be used for CRC detection.

In summary, MMBC-seq provides a highly sensitive multi-locus 5mC detection approach using plasma cfDNA: (i) it takes advantage of barcode counting and thermocycle-based linear amplification. Mutiple 5mC loci can be simultaneously amplified and accurately detected with extremely low copy numbers. This robust detection of multiple 5mC loci provides more comprehensive information to enhance the accuracy and comprehensiveness of clinical detection; (ii) simple probe design and straightforward workflow make MMBC simple and flexible to apply for detection of methylation markers in different diseases; (iii) MMBC-seq is low cost and is highly sensitive for detecting cfDNA from urine, stool, cerebrospinal fluid, and gDNA from fine needle biopsy and FFPE samples etc. Additional multiplex probes could be added to cover cancer markers from different tissue-of-origin as a universal 5mC panel for diagnosis of malignant human tumors.

B. Materials and Methods

1. DNA Materials Preparation

Human fully methylated genomic DNA (FMG) and unmethylated genomic DNA (UMG) were bought from EpiTect Control DNA and Control DNA set (Qiagen). Probes and model oligonucleotides in Table Si were ordered from IDT.

Blood from patients with colorectal cancer were collected prior to resection of the malignant lesion. The diagnosis was confirmed by surgical pathology after resection of the lesion. Controls were obtained from patients who underwent routine colonoscopies with no malignant or pre-malignant lesion found. Blood from controls was collected at time of the procedure or at next blood draw performed for routine purposes. This study is approved by the University of Chicago Institutional Review Board under protocol 10-209-A. Blood samples were collected in EDTA-tubes and were centrifuged for 1,350 g for 12 min at 4° C. twice, and 13,500 g for 5 min at 4° C. CfDNA was isolated from 1-2 mL plasma using QIAamp Circulating Nucleic Acid Kit.

All the required DNA materials were quantified by Qubit 3.0 (Life Invitrogen) and stored at ˜20° C.

2. Bisulfite Conversion

Bisulfite treatment was carried out with Invitrogen MethylCode™ Bisulfite Conversion Kit. Briefly, 20 μL gDNA or cfDNA was mixed with 130 μL CT conversion buffer, and incubated at 98° C. 10 min, 64° C. 2.5 hr, 4° C. hold. The mixture was purified by spin column with an on-column desulfionation. The converted DNA was eluted in 8 μL preheated (55° C.) DNase-free water and stored at −20° C. for later use.

3. Linear Amplification of Multiple Markers by Thermocycles

7 μL bisulfite treated DNA solution was transferred into a clean PCR tube and gently mixed with 1 μL 10× Multiplex Forward/Reverse Probe Mixture (100 nM per probes), 1 μL HiFi Taq Ligase (NEB) and 1 μL HiFi Taq Ligase 10× buffer (200 mM Tirs-HCl pH 8.3, 1500 mM KCl, 100 mM MgCl₂, 10 mM NAD and 1% Triton X-100, pH 8.5). Then, the reaction was subjected to the following condition: 180 sec at 95° C.; 180 cycles of 15 sec at 95° C., 90 sec 63° C. In this step, the ligation of forward and reverse probes can produce complete linear DNA template. To remove extra probes, 1 μL Exo I 20 U/μL, 1 μL Exo III 100 U/μL and 1 μL RecJf 30 U/μL were added, and incubated at 37° C. 4 hours followed by 15 min deactivation at 95° C.

4. MMBC qPCR Detection

qPCR assays were performed by Roche LightCyclerx® 96 qPCR machine. Product from linear amplification was mixed with 500 nM forward/reverse primers (P1/P2) and 2× Roche qPCR green master in qPCR 96 well plate. The reaction was subjected to 600 sec at 95° C.; 40 cycles of 20 sec at 95° C., 30 sec 60° C., 30 sec 72° C., followed by melting curve analysis ranging from 65° C. to 95° C., 0.2° C./s increment.

5. MMBC-Seq Library Construction

Preserved linear DNA templates were amplified by 50 μL PCR reaction, mixing 5-10 μL ligation product, 2.5 μL 10 μM of both forward and reverse primers (P1 and P2), 25 μL PCR 2× Master (KAPA HiFi polymerase) and DNase-free water. The first PCR program was: 45 sec at 98° C.; 24-27 cycles of 15 sec at 98° C., 30 sec 60° C., 30 sec 72° C. The number of PCR cycles depends on the amount of input DNA, so it should be titrated by qPCR first. The first PCR product was purified by gel extraction (80 bp-140 bp), then eluted in 30 μL DNase-free water and quantified by Qubit 3.0. Library construction was performed using KAPA HyperPlus Library Preparation Kit. Briefly, 10-20 ng gel-cut DNA in 20 μL ddH₂O was mixed with 2.8 μL End Repair & A-Tailling Buffer and 1.2 μL End Repair & A-Tailling Enzyme Mix, incubated at 65° C. 30 min. Next, 2 μL Nextflex adaptor, 2 μL ddH₂O, 12 μL Ligation Buffer and 4 μL DNA Ligase were added and incubated at 20° C. for 30 min. After post-ligation adapter clean-up, by using 0.9× Ampure XP Beads, the elution was secondary PCR-amplified for 5-7 cycles and purified by 0.9× Ampure XP Beads. The library (200 bp-260 bp) was sequenced using the NextSeq 500 platform.

6. MMBC-Seq Read Mapping and Data Analysis

An in-house program was designed to count the distinct barcodes of each locus.

First, reads were pre-processed by requiring both the P1 and P2 adaptors at 5′ and 3′ end. Then primer sequences beyond the adaptors were identified to determine the target genes. After each read was attributed to each target gene, the barcode sequences were counted. Identical barcode sequences were merged and the frequency of occurrence was recorded. Orphan barcodes which occurred only once were considered as being caused by sequencing error and discarded. The counting of distinct barcodes except the orphan ones was summed up to represent the copy number of each target.

Table S1
The sequence of probes and primers

| | | SEQ |
|---|---|---|
| | | ID |
| Name | Sequence | NO: |
| P1 primer | CTTCATCCTGCTGCTATGCCT | 1 |
| P2 primer | CCCAACTCCTCCCAGTCCTT | 2 |
| BMP3 Primer F | GTTTAATTTTCGGTTTCGTCGTC | 3 |
| BMP3 Primer R | CTCCCGACGTCGCTACG | 4 |
| NDRG4 Primer F | CGGTTTTCGTTCGTTTTTTCG | 5 |
| NDRG4 Primer R | GTAACTTCCGCCTTCTACGC | 6 |
| VIM Primer F | GGCGGTTCGGGTATCG | 7 |
| VIM Primer R | CGTAATCACGTAACTCCGACT | 8 |
| TFPI2 Primer F | TCGTTGGGTAAGGCGTTC | 9 |
| TFPI2 Primer R | AAACGAACACCCGAACCG | 10 |
| ACTB Primer F | TTTGTTTTTTTGATTAGGTGTTTAAGA | 11 |
| ACTB Primer R | CACCAACCTCATAACCTTATC | 12 |
| ACTB-1 | C*T*T*C*ATCCTGCTGCTATGCCTNNNNNNNNCACCAACCTC | 13 |
| | ATAACCTTATCACACAAACCAATATTAATAC | |
| ACTB-2 | /5Phos/CTACACCCACAACACTATCTTAAACACCTAATCAAAAA | 14 |
| | AACAAAAAGGACTGGGAGGAGT*T*G*G*G/3Phos/ | |
| VIM-1 | C*T*T*C*ATCCTGCTGCTATGCCTNNNNNNNNACGTAATCAC | 15 |
| | GTAACTCCGACTAAAACTCGACCG | |
| VIM-2 | /5Phos/ACTCGCGATACCCGAACCGCCGAACAAAGGACTGGG | 16 |
| | AGGAGT*T*G*G*G/3Phos/ | |
| NDRG4-1 | C*T*T*C*ATCCTGCTGCTATGCCTNNNNNNNNATAACTTCCGC | 17 |
| | CTTCTACGCGACTAAAATACCC | |
| NDRG4-2 | /5Phos/GATAAACGAACGAAAAAACGAACGAAAACCGAAGGA | 18 |
| | CTGGGAGGAGT*T*G*G*G/3Phos/ | |
| BMP3-1 | C*T*T*C*ATCCTGCTGCTATGCCTNNNNNNNNCTCCCGACGT | 19 |
| | CGCTACGAAACACTCCGAAAACG | |
| BMP3-2 | /5Phos/CAAAAAACCGACGACGAAACCGAAAATTAAACAAGG | 20 |
| | ACTGGGAGGAGT*T*G*G*G/3Phos/ | |
| TFPI2-1 | C*T*T*C*ATCCTGCTGCTATGCCTNNNNNNNNAAACGAACAC | 21 |
| | CCGAACCGCCTAAAACAAAAAACCGCG | |
| TFPI2-2 | /5Phos/CACCTCCTCCCGCCAAACGCTTTCTCGAACGCCTTA | 22 |
| | CCCAACGA/3Phos/ | |
| SEPT-F349-1 | C*T*T*C*ATCCTGCTGCTATGCCTNNNNNNNNCACCGCCGCC | 23 |
| | GCGCGCTCTAC | |
| SEPT-F349-2 | /5Phos/GCCTACAAAAATTAAACGACAACGCACGCGAAGGAC | 24 |
| | TGGGAGGAGT*T*G*G*G/3Phos/ | |
| SEPT-F486-1 | C*T*T*C*ATCCTGCTGCTATGCCTNNNNNNNNCCGAACGCCC | 25 |
| | CGCTACGACCAAATATAAAC | |
| SEPT-F486-2 | /5Phos/GAATATAAAAACCGAACCATAACGAAAAAAACGAACG | 26 |
| | CCAAGGACTGGGAGGAGT*T*G*G*G/3Phos/ | |
| SEPT-F567-1 | C*T*T*C*ATCCTGCTGCTATGCCTNNNNNNNNTCCTCCCCGC | 27 |
| | CTAACCCGC | |
| SEPT-F567-2 | /5Phos/GCCCAAAAAACGACGAAAAACGCGACCAAGGACTG | 28 |
| | GGAGGAGT*T*G*G*G/3Phos/ | |
| SEPT-F708-1 | C*T*T*C*ATCCTGCTGCTATGCCTNNNNNNNNCCCGAACGAA | 29 |
| | TCAAATTCCCGCACCC | |
| SEPT-F708-2 | /5Phos/GCACCGACCTCCCTATCTCGCACTAAGGACTGGGAG | 30 |
| | GAGT*T*G*G*G/3Phos/ | |
| SEPT-F767-1 | C*T*T*C*ATCCTGCTGCTATGCCTNNNNNNNNAAAACGAACG | 31 |
| | CCGACCCCTCCCCGC | |
| SEPT-F767-2 | /5Phos/GCGCTACGCCCCCGCCCCGAAGGACTGGGAGGAGT | 32 |
| | *T*G*G*G/3Phos/ | |
| SEPT-F856-1 | C*T*T*C*ATCCTGCTGCTATGCCTNNNNNNNNCGCGCTAACT | 33 |
| | AAAACGCCGCGCCC | |
| SEPT-F856-2 | /5Phos/GCGCTCCTACAATACAAAACTAACCGCCGAAAGGACT | 34 |
| | GGGAGGAGT*T*G*G*G/3Phos/ | |
| SEPT-F916-1 | C*T*T*C*ATCCTGCTGCTATGCCTNNNNNNNNACAACGAATC | 35 |
| | GCGCGAAAAACAACGACG | |
| SEPT-F916-2 | /5Phos/AAAAAACGCCCCCGACGAAACCCG | 36 |
| | AAGGACTGGGAGGAGT*T*G*G*G/3Phos/ | |
| SEPT-F1057-1 | C*T*T*C*ATCCTGCTGCTATGCCTNNNNNNNNCGTCCCGCGC | 37 |
| | CAAACCCACCC | |
| SEPT-F1057-2 | /5Phos/GCAAAATCCTCTCCAACACGTCCGCGACCGAAGGAC | 38 |
| | TGGGAGGAGT*T*G*G*G/3Phos/ | |
| SEPT-F1164-1 | C*T*T*C*ATCCTGCTGCTATGCCTNNNNNNNNCGTAAACGAC | 39 |
| | GCGAACACGAAACCGAAAACG | |
| SEPT-F1164-2 | /5Phos/CGCGACGCTCTCAACGAAAAAACGCCAAGGACTGG | 40 |
| | GAGGAGT*T*G*G*G/3Phos/ | |
| SEPT-F1442-1 | C*T*T*C*ATCCTGCTGCTATGCCTNNNNNNNNCGAAAATAAAA | 41 |
| | AACGACCTCCCTATCCCGTTACC | |
| SEPT-F1442-2 | /5Phos/GAATCCAAACGAAACCCGAAAACCGCCGAAGGACTG | 42 |
| | GGAGGAGT*T*G*G*G/3Phos/ | |
| SEPT-TFP12-1 | C*T*T*C*ATCCTGCTGCTATGCCTNNNNNNNNAAACGAACAC | 43 |
| | CCGAACCGCCTAAAACAAAAAACCGC | |
| SEPT-TFP12-2 | /5Phos/GCACCTCCTCCCGCCAAACGCTTTCTCGAACGAAGG | 44 |
| | ACTGGGAGGAGT*T*G*G*G/3Phos/ | |
| SEPT-F1563-1 | C*T*T*C*ATCCTGCTGCTATGCCTNNNNNNNNACCACGACCT | 45 |
| | AAACTATATCCGTTCGATACTCCCG | |
| SEPT-F1563-2 | /5Phos/ACCCACGAACTACACAACTTAACGACCCCGAAAGGA | 46 |
| | CTGGGAGGAGT*T*G*G*G/3Phos/ | |
| SEPT-F1841-1 | C*T*T*C*ATCCTGCTGCTATGCCTNNNNNNNNCGCGAACGCA | 47 |
| | AAACGCAAAACCCCAACACC | |
| SEPT-F1841-2 | /5Phos/GACAATCAAAAAAACGCAAAAAAACGCACGCACTCA | 48 |
| | AAGGACTGGGAGGAGTTGGG/3Phos/ | |
| SEPT-R608-1 | C*T*T*C*ATCCTGCTGCTATGCCTNNNNNNNNCCAAACGACA | 49 |
| | ACGACCTCCTCGAAAACTCG | |
| SEPT-R608-2 | /5Phos/CGAAACTACCTCGAAACTCTCCAAAACGCACAAAGG | 50 |
| | ACTGGGAGGAGT*T*G*G*G/3Phos/ | |
| SEPT-R785-1 | C*T*T*C*ATCCTGCTGCTATGCCTNNNNNNNNCGCTAAAAAC | 51 |
| | GCCGCGCGCCCCC | |
| SEPT-R785-2 | /5Phos/GACCCCGTACCCGCGCCGCCAAGGACTGGGAGGAG | 52 |
| | T*T*G*G*G/3Phos/ | |
| SEPT-R1035-1 | C*T*T*C*ATCCTGCTGCTATGCCTNNNNNNNNCCGCCGAAAA | 53 |
| | CGCTTCCTCGCC | |
| SEPT-R1035-2 | /5Phos/GCTACCCTCCGCGCGACCCGCTAAAGGACTGGGAG | 54 |
| | GAGT*T*G*G*G/3Phos/ | |
| TMEFF2-245-1 | C*T*T*C*ATCCTGCTGCTATGCCTNNNNNNNNNNNNACTCCT | 55 |
| | CTACATACGCCGCGAATAAATTACC | |
| TMEFF2-245-2 | /5Phos/GAAAACATCGACCGAACAACGACGTCCGAAGGACTG | 56 |
| | GGAGGAGT*T*G*G*G/3Phos/ | |
| TMEFF2-708-1 | C*T*T*C*ATCCTGCTGCTATGCCTNNNNNNNNNNNNCGTCCT | 57 |
| | ACTAACGACCGACGCTCCAAC | |
| TMEFF2-708-2 | /5Phos/GTACGAAAACGCGCCGCCTAAGGACTGGGAGGAGT* | 58 |
| | T*G*G*G/3Phos/ | |
| TMEFF2-805-1 | C*T*T*C*ATCCTGCTGCTATGCCTNNNNNNNNNNNNGCCCCT | 59 |
| | CTTCCGCGCGTAACCCC | |
| TMEFF2-805-2 | /5Phos/GAACCGCGAATACGACCGCGACAAGGACTGGGAGG | 60 |
| | AGT*T*G*G*G/3Phos/ | |
| TMEFF2-1010-1 | C*T*T*C*ATCCTGCTGCTATGCCTNNNNNNNNNNNNCGACCG | 61 |
| | CGAAAACCACAAATAAACTCGCTC | |
| TMEFF2-1010-2 | /5Phos/GCAAACGCTAACCCGAATAAAACTAAACGAAAGGACT | 62 |
| | GGGAGGAGT*T*G*G*G/3Phos/ | |
| TMEFF2-1214-1 | C*T*T*C*ATCCTGCTGCTATGCCTNNNNNNNNNNNNAAACGC | 63 |
| | CCCGCAACCCGACAACC | |
| TMEFF2-1214-2 | /5Phos/GCCTCTCGAACTCTACCGCCCGCAAGGACTGGGAG | 64 |
| | GAGT*T*G*G*G/3Phos/ | |
| TMEFF2-1500-1 | C*T*T*C*ATCCTGCTGCTATGCCTNNNNNNNNNNNNCGCGAA | 65 |
| | AACCGAACAACGAACTACTAAACATCCC | |
| TMEFF2-1500-2 | /5Phos/GCGAACGACGACAACAAAAACGACGACGAAAGGACT | 66 |
| | GGGAGGAGT*T*G*G*G/3Phos/ | |
| ALX4-112-1 | C*T*T*C*ATCCTGCTGCTATGCCTNNNNNNNNNNNNCGACTT | 67 |
| | AACCCGACGATCGCGACGAAATTCCTAAC | |
| ALX4-112-2 | /5Phos/GCAACCGCTTAAAACTTCGCATTAAAATCGAAACCGA | 68 |
| | AGGACTGGGAGGAGT*T*G*G*G/3Phos/ | |
| ALX4-776-1 | C*T*T*C*ATCCTGCTGCTATGCCTNNNNNNNNNNNNCGCTAC | 69 |
| | GAACGCACATAACCAAATAACGCCTAACAAAC | |
| ALX4-776-2 | /5Phos/GACGTCCGAATACAAAACGACGCTCTTACCG | 70 |
| | AAGGACTGGGAGGAGT*T*G*G*G/3Phos/ | |
| NGFR-204-1 | C*T*T*C*ATCCTGCTGCTATGCCTNNNNNNNNNNNNACCCGC | 71 |
| | GTCTAAACAACGTCTCTAACCAAC | |
| NGFR-204-2 | /5Phos/GACGTTAATCAACAAACGTACCCGCGATCGCTAAGGA | 72 |
| | CTGGGAGGAGT*T*G*G*G/3Phos/ | |
| NGFR-451-1 | C*T*T*C*ATCCTGCTGCTATGCCTNNNNNNNNNNNNCGCACA | 73 |
| | ACCATCCCAAACCGAACAACCGC | |
| NGFR-451-2 | /5Phos/GCGCCGAAACGAAAACGAAAACCGCAAAGGACTGG | 74 |
| | GAGGAGT*T*G*G*G/3Phos/ | |
| NGFR-557-1 | C*T*T*C*ATCCTGCTGCTATGCCTNNNNNNNNNNNNCCGACG | 75 |
| | CGACCCGCCAACC | |
| NGFR-557-2 | /5Phos/GACCCGCCGAAACGCGCTAAGGACTGGGAGGAGT*T | 76 |
| | *G*G*G/3Phos/ | |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the publications referred to throughout the specification, to the extent that they provide exemplary procedural or other details supplementary to those set forth herein, are specifically incorporated herein by reference.
Y. M. D. Lo, J. Zhang, T. N. Leung, T. K. Lau, A. M. Z. Chang, N. M. Hjelm, American Journal of Human Genetics 1999, 64, 218-224
A. K. Chan, R. W. Chiu, Y. D. Lo, Annals of clinical biochemistry 2003, 40, 122-130.
Y. M. D. Lo, J. Zhang, T. N. Leung, T. K. Lau, A. M. Z. Chang, N. M. Hjelm, American Journal of Human Genetics 1999, 64, 218-224
A. K. Chan, R. W. Chiu, Y. D. Lo, Annals of clinical biochemistry 2003, 40, 122-130.
C. Wong, F. Marass, S. Humphray, J. Hadfield, D. Bentley, T. M. Chin, J. D. Brenton, C. Caldas, N. Rosenfeld, Nature 2013, 497, 108-112;
L. A. D. Jr, A. Bardelli, Journal of Clinical Oncology 2014, 32, 579-586
G. Siravegna, S. Marsoni, S. Siena, A. Bardelli, Nat Rev Clin Oncol 2017, advance online publication.
R. Jaenisch, A. Bird, Nature Genetics 2003, 33, 245-254
T. H. Bestor, D. Bourc'his, Cold Spring Harbor Symposia on Quantitative Biology 2004, 69, 381-387
R. J. Klose, A. P. Bird, Trends in Biochemical Sciences 2006, 31, 89-97.
H. G. Hernandez, M. Y. Tse, S. C. Pang, H. Arboleda, D. A. Forero, Biotechniques 2013, 55, 181-197.
T. R. Church, M. Wandell, C. Lofton-Day, S. J. Mongin, M. Burger, S. R. Payne, E. Castaños-Vélez, B. A. Blumenstein, T. Rösch, N. Osborn, Gut 2014, 63, 317-325
J. D. Warren, W. Xiong, A. M. Bunker, C. P. Vaughn, L. V. Furtado, W. L. Roberts, J. C. Fang, W. S. Samowitz, K. A. Heichman, BMC medicine 2011, 9, 133.
K. Sun, P. Y. Jiang, K. C. A. Chan, J. Wong, Y. K. Y. Cheng, R. H. S. Liang, W. K. Chang, E. S. K. Ma, S. L. Chan, S. H. Cheng, R. W. Y. Chan, Y. K. Tong, S. S. M. Ng, R. S. M. Wong, D. S. C. Hui, T. N. Leung, T. Y. Leung, P. B. S. Lai, R. W. K. Chiu, Y. M. D. Lo, Proceedings of the National Academy of Sciences of the United States of America. 2015, 112, E5503-E5512
S. C. Guo, D. Diep, N. Plongthongkum, H. L. Fung, K. Zhang, K. Zhang, Nature Genetics 2017, 49, 635-+
K. C. A. Chan, P. Jiang, C. W. M. Chan, K. Sun, J. Wong, E. P. Hui, S. L. Chan, W. C. Chan, D. S. C. Hui, S. S. M. Ng, H. L. Y. Chan, C. S. C. Wong, B. B. Y. Ma, A. T. C. Chan, P. B. S. Lai, H. Sun, R. W. K. Chiu, Y. M. D. Lo, Proceedings of the National Academy of Sciences of the United States of America 2013, 110, 18761-18768.
L. Wen, J. Li, H. Guo, X. Liu, S. Zheng, D. Zhang, W. Zhu, J. Qu, L. Guo, D. Du, Cell research 2015, 25, 1250-1264.
K. Warton, V. Lin, T. Navin, N. J. Armstrong, W. Kaplan, K. Ying, B. Gloss, H. Mangs, S. S. Nair, N. F. Hacker, BMC genomics 2014, 15, 476.
F. Vaca-Paniagua, J. Oliver, A. N. da Costa, P. Merle, J. McKay, Z. Herceg, R. Holmila, Epigenomics 2015, 7, 353-362
R. Lehmann-Werman, D. Neiman, H. Zemmour, J. Moss, J. Magenheim, A. Vaknin-Dembinsky, S. Rubertsson, B. Nellgard, K. Blennow, H. Zetterberg, Proceedings of the National Academy of Sciences 2016, 113, E1826-E1834
Y. Korshunova, R. K. Maloney, N. Lakey, R. W. Citek, B. Bacher, A. Budiman, J. M. Ordway, W. R. McCombie, J. Leon, J. A. Jeddeloh, Genome research 2008, 18, 19-29.
J. Deng, R. Shoemaker, B. Xie, A. Gore, E. M. LeProust, J. Antosiewicz-Bourget, D. Egli, N. Maherali, I.-H. Park, J. Yu, Nature biotechnology 2009, 27, 353-360.
G. K. Fu, J. Hu, P.-H. Wang, S. P. Fodor, Proceedings of the National Academy of Sciences 2011, 108, 9026-9031; b) K. Shiroguchi, T. Z. Jia, P. A. Sims, X. S. Xie, Proceedings of the National Academy of Sciences 2012, 109, 1347-1352.
W.-D. Chen, Z. J. Han, J. Skoletsky, J. Olson, J. Sah, L. Myeroff, P. Platzer, S. Lu, D. Dawson, J. Willis, Journal of the National Cancer Institute 2005, 97, 1124-1132.
H. Zou, H. Allawi, X. Cao, M. Domanico, J. Harrington, W. R. Taylor, T. Yab, D. A. Ahlquist, G. Lidgard, Clinical chemistry 2012, 58, 375-383.
L. Song, Y. Li, Advances in clinical chemistry 2015, 72, 171-204
M. Galanopoulos, N. Tsoukalas, I. S. Papanikolaou, M. Tolia, M. Gazouli, G. J. Mantzaris, World Journal of Gastrointestinal Oncology 2017, 9, 142-152
F. Model, N. Osborn, D. Ahlquist, R. Gruetzmann, B. Molnar, F. Sipos, O. Galamb, C. Pilarsky, H.-D. Saeger, Z. Tulassay, Molecular cancer research 2007, 5, 153-163
C. Lofton-Day, F. Model, T. DeVos, R. Tetzner, J. Distler, M. Schuster, X. Song, R. Lesche, V. Liebenberg, M. Ebert, Clinical chemistry 2008, 54, 414-423.

1. A method for detecting methylated or unmethylated cytosines in one or more regions of target nucleic acids, the method comprising
i) combining a solution comprising the target nucleic acids with a deaminating agent to convert unmethylated cytosines in the target nucleic acids to uracils;
ii) next contacting the solution with at least two probes under conditions that allow for the hybridization of the two probes to one target nucleic acid region; wherein a terminal end from each probe hybridizes adjacently to the target nucleic acid region;
iii) contacting the solution comprising the hybridized probes and target nucleic acids with a ligase under conditions that allow for the ligation of the terminal ends of the adjacently hybridized probes; and
iv) detecting the adjacently hybridized ligated probes in the solution.

2. The method of claim 1, wherein at least one of the probes comprise a unique identifier (UID).

3. The method of claim 2, wherein the UID is specific for the target nucleic acid region.

4. The method of claim 2 or 3, wherein the UID is on the tail of the probe.

5. The method of any one of claims 1-4, wherein the method further comprises a denaturing step.

6. The method of claim 5, wherein ii comprises: iia) incubating the solution under conditions sufficient for the denaturation of nucleic acid and iib) next contacting the solution with at least two probes under conditions that allow for the hybridization of the two probes to one target nucleic acid region; wherein a terminal end from each probe hybridizes adjacently to the target nucleic acid region.

7. The method of any one of claims 1-6, wherein the method comprises repeating steps ii and/or iii more than one time.

8. The method of claim 7, wherein ii and iii is repeated at least 2 times prior to detection of the adjacently hybridized ligated probes.

9. The method of any one of claims 1-8, wherein the deaminating agent comprises bisulfite.

10. The method of any one of claims 1-9, wherein one or both of the probes comprise a primer binding site.

11. The method of claim 10, wherein the primer binding site is on the tail of the probe.

12. The method of any one of claims 1-11, wherein detection of the adjacently hybridized ligated probes in the solution comprises linear detection.

13. The method of any one of claims 1-12, wherein the method further comprises PCR amplification of the adjacently hybridized ligated probes.

14. The method of any one of claims 1-13, wherein detecting the adjacently hybridized ligated probes comprises quantitative PCR.

15. The method of any one of claims 1-14, wherein the method further comprises ligation of one or more adaptors to the adjacently hybridized ligated probes.

16. The method of any one of claims 1-15, wherein detecting the ligated adjacently hybridized probes comprises sequencing the probes.

17. The method of any one of claims 1-16, wherein at least two target regions are detected.

18. The method of claim 17, wherein the at least two target regions are detected from target nucleic acids in the same solution.

19. The method of claim 17 or 18, wherein ii) comprises contacting the solution with at least four probes under conditions that allow for the hybridization of the two probes to one target nucleic acid region and two probes to a different target nucleic acid region.

20. The method of any one of claims 1-19, wherein the target nucleic acid region comprises at least 40 contiguous nucleic acids.

21. The method of any one of claims 1-20, wherein one or more of the probes comprises a 3′ or 5′ tail.

22. The method of claim 21, wherein the tail comprises phosphorothioate-modified nucleic acids.

23. The method of claim 22, wherein the tail comprises at least 2 phosphorothioate-modified nucleic acids.

24. The method of any one of claims 1-23, wherein one or more of the probes comprise a terminal cap.

25. The method of claim 24, wherein the terminal cap is at the terminal end of a tail.

26. The method of any one of claims 1-25, wherein the method further comprises contacting the solution comprising the ligated probes with an exonuclease.

27. The method of any one of claims 1-26, wherein steps i)-iv) are performed in sequential order.

28. The method of any one of claims 1-27, wherein the probe hybridizes to fully methylated target nucleic acids.

29. The method of any one of claims 1-28, wherein the solution comprising the target nucleic acids comprises less than 1 ng of nucleic acids.

30. The method of any one of claims 1-29, wherein the method further comprises:
i) combining a solution comprising the control nucleic acids with a deaminating agent to convert unmethylated cytosines to uracils;
ii) next contacting the solution with at least two control probes under conditions that allow for the hybridization of the two probes to one control nucleic acid region; wherein a terminal end from each probe hybridizes adjacently to the control nucleic acid region;
iii) contacting the solution comprising the hybridized control probes and control nucleic acids with a ligase under conditions that allow for the ligation of the terminal ends of the adjacently hybridized control probes; and
iv) detecting the adjacently hybridized ligated control probes in the solution.

31. The method of claim 29 or 30, wherein the control probes hybridize to unmethylated, deaminated control nucleic acids.

32. The method of claim 29 or 30, wherein the control probes hybridize to methylated control nucleic acids.

33. The method of claim 29 or 30, wherein the control probes hybridize to partially methylated nucleic acids.

34. The method of any one of claims 30-33, wherein the control nucleic acids are in the same solution as the target nucleic acids.

35. The method of any one of claims 30-33, wherein the control nucleic acids are in a different solution as the target nucleic acids.

36. The method of any one of claims 30-35, wherein the control nucleic acids comprise a known percentage of fully methylated control target nucleic acids.

37. The method of claim 36, wherein the method further comprises construction of a calibration curve.

38. The method of any one of claims 1-37, wherein the ligase comprises a thermostable ligase.

39. The method of any one of claims 1-38, wherein the method further comprises library construction from the hybridized probes.

40. The method of any one of claims 1-39, wherein the target and/or control nucleic acids comprise cell free DNA.

41. The method of any one of claims 1-40, wherein the target and/or control nucleic acids are isolated from a cell or population of cells.

42. The method of any one of claims 1-41, wherein the number of CpGs in each target region or control target region is 5-12.

43. The method of any one of claims 1-42, wherein the method further comprises determining the total amount of target region.

44. The method of claim 43, wherein determining the total amount of target region comprises performing a non-deamination control sample using target probes that hybridize to undeaminated target region.

45. The method of any one of claims 1-44, wherein the target and/or control nucleic acids are isolated from a serum, urine, stool, cerebrospinal fluid, biopsy, fine needle biopsy, genomic DNA, frozen, or formalin-fixed, paraffin-embedded sample.

46. The method of any one of claims 1-45, wherein the target and/or control nucleic acids are isolated from a sample from a patient with a disease or disorder.

47. The method of claim 46, wherein the disease or disorder comprises autoimmunity or cancer.

48. A kit comprising at least one probe that hybridizes to a target region, wherein the probe hybridizes to fully methylated CpG target region that has been deaminated.

49. The kit of claim 48, wherein the kit comprises at least two probes.

50. The kit of claim 48 or 49, further comprising a deaminating agent, ligase, polymerase, or exonuclease.

51. The kit of any one of claims 48-50, wherein the deaminating agent comprises bisulfite.

52. The kit of any one of claims 48-51, wherein the kit comprises reagents for isolating target nucleic acids from a biological sample, for amplifying nucleic acids from a sample by PCR, and/or for amplifying nucleic acids by real-time or quantitative.

53. The kit of any one of claims 48-52, wherein the kit further comprises ligase.

54. The kit of any one of claims 48-53, wherein at least one of the probes comprise a unique identifier (UID).

55. The kit of any one of claims 48-54, wherein the UID is specific for the target nucleic acid region.

56. The kit of claim 54 or 55, wherein the UID is on the tail of the probe.

57. The kit of any one of claims 48-56, wherein one or both of the probes comprise a primer binding site.

58. The kit of claim 57, wherein the primer binding site is on the tail of the probe.

59. The kit of any one of claims 48-58, wherein one or more of the probes comprises a 3′ or 5′ tail.

60. The kit of claim 59, wherein the tail comprises phosphorothioate-modified nucleic acids.

61. The kit of claim 60, wherein the tail comprises at least 2 phosphorothioate-modified nucleic acids.

62. The kit of any one of claims 48-61, wherein one or more of the probes comprise a terminal cap.

63. The kit of claim 62, wherein the terminal cap is at the terminal end of a tail.

64. A nucleic acid probe comprising a UID and a hybridization region that hybridizes to fully methylated DNA of a target DNA.

65. The nucleic acid probe of claim 64, wherein the probe comprises DNA.

66. The nucleic acid probe of claim 64 or 65, wherein the UID is specific for the target nucleic acid region.

67. The nucleic acid probe of any one of claims 64-66, wherein the UID is on the tail of the probe.

68. The nucleic acid probe of any one of claims 65-67, further comprising at least one primer binding site.

69. The probe of claim 68, wherein the at least one primer binding site is on the tail of the probe.

70. The probe of any one of claims 64-69, wherein the probe comprises a 3′ or 5′ tail.

71. The probe of claim 70, wherein the tail comprises phosphorothioate-modified nucleic acids.

72. The probe of claim 71, wherein the tail comprises at least 2 phosphorothioate-modified nucleic acids.

73. The probe of any one of claims 64-72, wherein the probe comprises a terminal cap.

74. The probe of claim 73, wherein the terminal cap is at the terminal end of a tail.

75. The probe of any one of claims 64-74, wherein the probe hybridizes to a hypermethylation-associated disease region.
